SEQUENCE LISTING (50 sequences)

SEQ ID NO: 1 - protein, 139 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPR

SEQ ID NO: 2 - protein, 135 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDLLGYTDKLIFGKGTRVTVEPR

SEQ ID NO: 3 - protein, 137 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPR

SEQ ID NO: 4 - DNA, 879 bp, synthetic construct
  source 1..879: mol_type = other DNA
atggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacaccctgggcatgggcggcgagtacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 5 - DNA, 867 bp, synthetic construct
  source 1..867: mol_type = other DNA
atggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacctgctgggctacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 6 - DNA, 873 bp, synthetic construct
  source 1..873: mol_type = other DNA
atggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacgccctgaagagaaccgacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 7 - protein, 8 aa, synthetic construct
TLGMGGEY

SEQ ID NO: 8 - protein, 4 aa, synthetic construct
LLGY

SEQ ID NO: 9 - protein, 6 aa, synthetic construct
ALKRTD

SEQ ID NO: 10 - protein, 16 aa, synthetic construct
ACDTLGMGGEYTDKLI

SEQ ID NO: 11 - protein, 12 aa, synthetic construct
ACDLLGYTDKLI

SEQ ID NO: 12 - protein, 14 aa, synthetic construct
ACDALKRTDTDKLI

SEQ ID NO: 13 - protein, 142 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIIT

SEQ ID NO: 14 - protein, 140 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEEELGKKIKVFGPGTKLIIT

SEQ ID NO: 15 - protein, 141 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIIT

SEQ ID NO: 16 - DNA, 948 bp, synthetic construct
  source 1..948: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggaggcccagcaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctga

SEQ ID NO: 17 - DNA, 943 bp, synthetic construct
  source 1..943: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggaggaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctgag

SEQ ID NO: 18 - DNA, 942 bp, synthetic construct
  source 1..942: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggagatccaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagc

SEQ ID NO: 19-20 - skipped (2 entries)

SEQ ID NO: 21 - protein, 15 aa, synthetic construct
ALWEAQQELGKKIKV

SEQ ID NO: 22 - protein, 14 aa, synthetic construct
ALWEIQELGKKIKV

SEQ ID NO: 23 - protein, 153 aa, synthetic construct
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 24 - protein, 173 aa, synthetic construct
DKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 25 - protein, 121 aa, synthetic construct
SAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPR

SEQ ID NO: 26 - protein, 117 aa, synthetic construct
SAIELVPEHQTVPVSIGVPATLRCSMKGEAIGNYYINWYRKTQGNTMTFIYREKDIYGPGFKDNFQGDIDIAKNLAVLKILAPSERDEGSYYCACDLLGYTDKLIFGKGTRVTVEPR

SEQ ID NO: 27 - protein, 119 aa, synthetic construct
SAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPR

SEQ ID NO: 28 - protein, 123 aa, synthetic construct
GAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIIT

SEQ ID NO: 29 - protein, 121 aa, synthetic construct
GAGHLEQPQISSTKTLSKTARLECVVSGITISATSVYWYRERPGEVIQFLVSISYDGTVRKESGIPSGKFEVDRIPETSTSTLTIHNVEKQDIATYYCALWEEELGKKIKVFGPGTKLIIT

SEQ ID NO: 30 - protein, 122 aa, synthetic construct
GAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIIT

SEQ ID NO: 31 - protein, 292 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 32 - protein, 288 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDLLGYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 33 - protein, 290 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 34 - protein, 315 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 35 - protein, 313 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEEELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 36 - protein, 314 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 37 - protein, 625 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGEGRGSLLTCGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 38 - protein, 626 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGEGRGSLLTCGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 39 - protein, 627 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGEGRGSLLTCGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 40 - protein, 624 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGEGRGSLLTCGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDLLGYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 41 - DNA, 1878 bp, synthetic construct
  source 1..1878: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggagatccaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcggcagcggcgaaggccgcggcagcctgctgacctgcggcgatgtggaagaaaaccctggcccgatggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacgccctgaagagaaccgacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 42 - DNA, 1884 bp, synthetic construct
  source 1..1884: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggaggcccagcaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctgaggcagcggcgaaggccgcggcagcctgctgacctgcggcgatgtggaagaaaaccctggcccgatggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacgccctgaagagaaccgacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 43 - DNA, 1884 bp, synthetic construct
  source 1..1884: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggagatccaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcggcagcggcgaaggccgcggcagcctgctgacctgcggcgatgtggaagaaaaccctggcccgatggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacaccctgggcatgggcggcgagtacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 44 - DNA, 1878 bp, synthetic construct
  source 1..1878: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggaggcccagcaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagctgaggcagcggcgaaggccgcggcagcctgctgacctgcggcgatgtggaagaaaaccctggcccgatggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacctgctgggctacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 45 - DNA, 367 bp, synthetic construct
  source 1..367: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcact

SEQ ID NO: 46 - DNA, 594 bp, synthetic construct
  source 1..594: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 47 - DNA, 1182 bp, synthetic construct
  source 1..1182: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 48 - DNA, 399 bp, synthetic construct
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 49 - DNA, 6539 bp, synthetic construct
  source 1..6539: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatgcatgctgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcactagcggccctaatacgactcactatagcccggctcgaggctaggcgcgaattcgccacccggatccacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 50 - DNA, 1878 bp, synthetic construct; identical to SEQ ID NO: 41
  source 1..1878: mol_type = other DNA

(19) United States
(12) Patent Application Publication
(10) Pub. No.: US 2024/0043496 A1
(43) Pub. Date: Feb. 8, 2024

(54) DELTA T-CELL OR GAMMA RECEPTOR CHAINS OR PARTS THEREOF FOR TREATING OVARIAN CANCER

(71) Applicant: Gadeta B.V., Utrecht (NL)

(72) Inventor: Marleen van Loenen, Utrecht (NL)

(21) Appl. No.: 18/359,420

(22) Filed: Jul. 26, 2023

Related U.S. Application Data
(60) Provisional application No. 63/392,428, filed on Jul. 26, 2022.

(51) Int. Cl. C07K 14/725; A61K 45/06; A61P 35/00

(52) U.S. Cl. CPC C07K 14/7051; A61K 45/06; A61P 35/00; A61K 38/00

(57) ABSTRACT

Provided herein are methods of treating ovarian cancer using compositions comprising a γ9δ2T-cell receptor or a fragment thereof, a nucleotide sequence encoding it, a vector comprising said nucleotide sequence, or an engineered immune responsive cell that comprises or expresses an exogenous γ9δ2T-cell receptor or a functional fragment thereof.

### CROSS REFERENCE TO RELATED APPLICATION

This application claims the benefit of U.S. Provisional Application No. 63/392,428 filed on Jul. 26, 2022, the disclosure of which is hereby incorporated by reference in its entirety.

### SEQUENCE LISTING

The instant application contains a Sequence Listing which has been submitted electronically in XML format and is hereby incorporated by reference in its entirety for all purposes. The XML copy, created on Jul. 25, 2023, is referred to as 115436-766532_GDT-014-SequenceListingxml and is 72 kilobytes in size.

### FIELD OF THE INVENTION

The present disclosure relates to δT-cell (or γT-cell) receptors chains or parts thereof, or γδTCR, or cells comprising or expressing them, and their use in treating ovarian cancer.

### BACKGROUND OF THE INVENTION

Ovarian cancer accounts for a fifth of cancer deaths among women and accounts for higher mortality than any other cancer of the female reproductive system. One in 78 women get ovarian cancer during their lifetime. Although progress has been made in its treatment by improved surgical procedures and the introduction of improved platinum-based chemotherapeutic regimens, the overall 5-year survival is only about 29% in advanced stage disease, mostly due to diagnoses occurring at an advanced stage, and to intrinsic and acquired resistance to platinum-based chemotherapy.

Adoptive immunotherapy provides a viable alternative to traditional treatment methods against multiple cancer types, including ovarian cancer. Our immune system utilizes different lines of defense to protect us from infections as well as cancer. In order to cover the magnitude of potential invaders and internal threats our adaptive immune system has the possibility to raise up to 10¹⁶ αβTCR combinations as well as 10¹¹ variations in immunoglobulins.

Among all immune receptor chains, TCR δs have the highest potential diversity in the CDR3 loop (approximately 10¹⁶ combinations for murine TCR δ) owing to the presence of multiple D gene segments (two in mice, three in human, and up to five in cattle) that can join together. Each D gene segment can be read in all three open reading frames, and N nucleotides can be inserted into the junctions of the joining segments. Thus, despite the limited diversity at the VJ junctions of TCR γ-chains, the potential diversity generated at the combined CDR3 junctions (approximately 10¹⁸ combinations) is still higher than that of αβTCRs (˜10¹⁶) and immunoglobulins (˜10¹¹).

Ways of utilizing this diversity to develop effective anti-cancer therapeutics against ovarian and other cancer types is therefore a highly consequential endeavor.

### SUMMARY OF THE INVENTION

In some aspects the current disclosure encompasses a method of treating ovarian cancer in a subject in need thereof, the method comprising: administering to the subject a composition comprising a γ9δ2T-cell receptor or a fragment thereof, a nucleotide sequence encoding it, a vector comprising said nucleotide sequence, or an engineered immune responsive cell that comprises or expresses an exogenous γ9δ2T-cell receptor or a functional fragment thereof. In some aspect the composition is a pharmaceutical composition. In some aspects the composition further comprises a pharmaceutically acceptable carrier and/or excipient.

In some aspects of the disclosed method, the engineered immune responsive cell is a αβ T-cell engineered to express said exogenous γ9δ2T-cell receptor or a functional fragment thereof. In some aspects the current disclosure also encompasses a method of treating ovarian cancer in a subject in need thereof, the method comprising: contacting an ovarian tumor cell with the compositions disclosed herein. In some aspects the compositions comprise a γ9δ2T-cell receptor or fragment thereof comprises a polypeptide (e.g., a first polypeptide) having at least 60%, 70%, 80%, 90%, 95%, or 100% identity or similarity with an amino acid sequence selected from SEQ ID NOS: 13-15, 19-22, 24, 28-30, 34-36, and/or a polypeptide (e.g., a second polypeptide) having at least 60%, 70%, 80%, 90%, 95%, or 100% identity or similarity with an amino acid sequence selected from SEQ ID NOS: 1-3, 7-12, 23, 25-27, 31-33. In some aspects the current disclosure also encompasses compositions comprising a nucleotide sequence encoding a polypeptide (e.g., a first polypeptide) having at least 60%, 70%, 80%, 90%, 95%, or 100% identity or similarity with an amino acid sequence selected from SEQ ID NOS: 13-15, 19-22, 24, 28-30, 34-36, and/or a nucleotide sequence encoding a polypeptide (e.g., a second polypeptide) having at least 60%, 70%, 80%, 90%, 95%, or 100% identity or similarity with an amino acid sequence selected from SEQ ID NOS: 1-3, 7-12, 23, 25-27, 31-33. In some aspects of the method, the γ9δ2T-cell receptor comprises a γ9-T-cell receptor chain comprising a γ9-CDR3 region having a sequence selected from any one of SEQ ID NOS: 19-24 and a δ2-CDR3 region having a sequence selected from SEQ ID NOS: 7-12.

In some aspects, the composition comprises a population of said αβ T-cells engineered to express said γ9δ2T-cell receptor.

In some aspects the composition comprises γ9δ2T-cell receptor comprising an γ9-T-cell receptor chain comprising an amino acid sequence having at least 70% sequence identity or similarity to any one of SEQ ID NOS: 13-15, 19-22, 28-30 or 34-36; and a δ2-T-cell receptor chain comprising an amino acid sequence having at least 70% sequence identity or similarity to any one of SEQ ID NOS: 1-3, 7-12, 25-27 or 31-33. In some aspects the γ9δ2T-cell receptor comprises a γ9-T-cell receptor chain comprising an amino acid sequence having at least 80% sequence identity or similarity to any one of SEQ ID NOS: 13-15, 19-22, 28-30 or 34-36; and a δ2-T-cell receptor chain comprising an amino acid sequence having at least 80% sequence identity or similarity to any one of SEQ ID NOS: 1-3, 7-12, 25-27 or 31-33. In some aspects the γ9δ2T-cell receptor comprises a γ9-T-cell receptor chain comprising an amino acid sequence having at least 90% sequence identity or similarity to any one of SEQ ID NOS: 13-15, 19-22, 28-30 or 34-36; and a δ2-T-cell receptor chain comprising an amino acid sequence having at least 90% sequence identity or similarity to any one of SEQ ID NOS: 1-3, 7-12, 25-27 or 31-33. In some aspects the γ9δ2T-cell receptor comprises a γ9-T-cell receptor chain comprising an amino acid sequence having at least 95% sequence identity or similarity to any one of SEQ ID NOS: 13-15, 19-22, 28-30 or 34-36; and a δ2-T-cell receptor chain comprising an amino acid sequence having at least 95% sequence identity or similarity to any one of SEQ ID NOS: 1-3, 7-12, 25-27 or 31-33.

In some aspects of the method, the pharmaceutical composition is administered intravenously to the subject. In some aspects of the method, administering the composition reduces number of ovarian cancer cells. In some aspects the composition is cytotoxic for ovarian cancer cells. In some aspects administering the composition reduces ovarian cancer growth. In some aspects the current disclosure also encompasses administering to the subject an additional anti-cancer therapy either before, after or with administering the disclosed composition. In some aspects the additional anti-cancer therapy comprises any one of surgery, radiation, chemotherapy or a combination thereof. In some aspects the subject is a human.

### BRIEF DESCRIPTION OF THE FIGURES

FIG. 1. Plasmid map of pLenti6.3.

FIG. 2A. γ9δ2 TCR displays tumor reactivity against OVCAR-3 ovarian carcinoma cell line. OVCAR-3 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate (PAM; closed circles). Cytotoxicity was measured by xCELLigence and plotted as percentage of cytolysis relative to maximum cytolysis induced by treatment of the target cells with the detergent Triton-X-100. Median percentage cytolysis of three donors is provided, each performed in triplicate. **P<0.01

FIG. 2B. γ9δ2 TCR displays tumor reactivity against OV-90 ovarian carcinoma cell line. OV-90 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate (PAM; closed circles). Cytotoxicity was measured by xCELLigence and plotted as percentage of cytolysis relative to maximum cytolysis induced by treatment of the target cells with the detergent Triton-X-100. Median percentage cytolysis of three donors is provided, each performed in triplicate. **P<0.01

FIG. 2C. γ9δ2 TCR displays tumor reactivity against COV504 ovarian carcinoma cell line. COV504 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate (PAM; closed circles). Cytotoxicity was measured by xCELLigence and plotted as percentage of cytolysis relative to maximum cytolysis induced by treatment of the target cells with the detergent Triton-X-100. Mean±SD percentage cytolysis of three donors is provided, each performed in triplicate. *P<0.05

FIG. 2D. γ9δ2 TCR displays tumor reactivity against CAOV-3 ovarian carcinoma cell line. CAOV-3 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate (PAM; closed circles). Cytotoxicity was measured by xCELLigence and plotted as percentage of cytolysis relative to maximum cytolysis induced by treatment of the target cells with the detergent Triton-X-100. Mean±SD percentage cytolysis of three donors is provided, each performed in triplicate. *P<0.05

FIG. 2E. γ9δ2 TCR displays tumor reactivity against SK-OV-3 ovarian carcinoma cell line. SK-OV-3 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate (PAM; closed circles). Cytotoxicity was measured by xCELLigence and plotted as percentage of cytolysis relative to maximum cytolysis induced by treatment of the target cells with the detergent Triton-X-100. Mean±SD percentage cytolysis of three donors is provided, each performed in triplicate. *P<0.05

FIG. 3A. γ9δ2 TCR displays tumor reactivity against OVCAR-3 ovarian carcinoma cell line, as measured by IFN-γ. OVCAR-3 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate, (PAM; closed circles). The level of IFN-γ released into the supernatants were measured by ELISA. Data depicted show the mean±SD of 3 independent donors, each performed in triplicate.

FIG. 3B. γ9δ2 TCR displays tumor reactivity against OV-90 ovarian carcinoma cell line, as measured by IFN-γ. OV-90 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate, (PAM; closed circles). The level of IFN-γ released into the supernatants were measured by ELISA. Data depicted show the mean±SD of 3 independent donors, each performed in triplicate.

FIG. 3C. γ9δ2 TCR displays tumor reactivity against COV504 ovarian carcinoma cell line, as measured by IFN-γ. COV504 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate, (PAM; closed circles). The level of IFN-γ released into the supernatants were measured by ELISA. Data depicted show the mean±SD of 3 independent donors, each performed in triplicate.

FIG. 3D. γ9δ2 TCR displays tumor reactivity against CAOV-3 ovarian carcinoma cell line, as measured by IFN-γ. CAOV-3 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate, (PAM; closed circles). The level of IFN-γ released into the supernatants were measured by ELISA. Data depicted show the mean±SD of 3 independent donors, each performed in triplicate.

FIG. 3E. γ9δ2 TCR displays tumor reactivity against SK-OV-3 ovarian carcinoma cell line, as measured by IFN-γ. SK-OV-3 tumor cell line was co-cultured for 48 hr with TEGs expressing γδTCR CL5 (CDR3 regions represented by SEQ ID NOs: 12, 22) or untransduced matched αβT-cells (negative control) or endogenous γ9δ2 T cells, at effector to target (E:T) ratio of 1:1 in the absence (open circles) or presence of 10 μM pamidronate, (PAM; closed circles). The level of IFN-γ released into the supernatants were measured by ELISA. Data depicted show the mean±SD of 3 independent donors, each performed in triplicate.

FIG. 4A. Dose dependent tumor reactivity as measured by cytotoxicity of CL5. CAOV-3 tumour cell lines were co-cultured for 24 hr with TEGs expressing γδTCR CL5 (CDR3 regions represented by SEQ ID NOs: 12, 22) or untransduced matched αβT-cells (negative control), at effector to target (E:T) ratio of 1:1 in the presence of increasing concentration of pamidronate (PAM) up to 30 μM. Cytotoxicity was measured by xCELLigence and plotted as percentage of cytolysis relative to maximum cytolysis induced by treatment of the target cells with the detergent Triton-X-100. Representative has been depicted (N=3).

FIG. 4B. Dose dependent tumor reactivity as measured by IFN-γ release induced by CL5. CAOV-3 tumour cell lines were co-cultured for 24 hr with TEGs expressing γδTCR CL5 (CDR3 regions represented by SEQ ID NOs: 12, 22) or untransduced matched αβT-cells (negative control), at effector to target (E:T) ratio of 1:1 in the presence of increasing concentration of pamidronate (PAM) up to 30 μM. The levels of IFN-γ released into the supernatants were measured by ELISA. Representative has been depicted (N=3).

FIG. 5A. TCR displays tumour reactivity against patient-derived ovarian tumor organoids. Patient-derived ovarian tumor organoids were co-cultured with 0.5E6 TEGs expressing γδTCR CL5 (CDR3 regions represented by SEQ ID NOs: 12, 22) or untransduced matched αβT-cells (negative control) for 72 hours in the presence of 10 μM PAM. IFN-γ released into the supernatants were measured by ELISA. Data depicted show one dot for each patient-derived organoid, measured in two independent experiments each in triplicate.

FIG. 5B. TCR displays tumour reactivity against patient-derived colon tumor organoids. Patient-derived colon tumor organoids were co-cultured with 0.5E6 TEGs expressing γδTCR CL5 (CDR3 regions represented by SEQ ID NOs: 12, 22) or untransduced matched αβT-cells (negative control) for 72 hours in the presence of 10 μM PAM. IFN-γ released into the supernatants were measured by ELISA. Data depicted show one dot for each patient-derived organoid, measured in two independent experiments each in triplicate.

FIG. 5C. TCR displays tumour reactivity against patient-derived breast tumor organoids. Patient-derived breast tumor organoids were co-cultured with 0.5E6 TEGs expressing γδTCR CL5 (CDR3 regions represented by SEQ ID NOs: 12, 22) or untransduced matched αβT-cells (negative control) for 72 hours in the presence of 10 μM PAM. IFN-γ released into the supernatants were measured by ELISA. Data depicted show one dot for each patient-derived organoid, measured in two independent experiments each in triplicate.

FIG. 6A shows total tumoroid area in co-cultures of ascites and effector cells from one donor. Effector cells are αβ T cells that are TRAC knock-out either expressing the GDT002 receptor or not (UNTR) in the presences or absence of Zoledronic acid. Total tumoroid area in mm³ was measured and normalized against UNTR-ZOL.

FIG. 6B shows total tumoroid area in co-cultures of ascites and effector cells from one donor. Effector cells are αβ T cells that are TRAC knock-out either expressing the GDT002 receptor or not (UNTR) in the presences or absence of Zoledronic acid. Total tumoroid area in mm³ was measured and normalized against UNTR-ZOL.

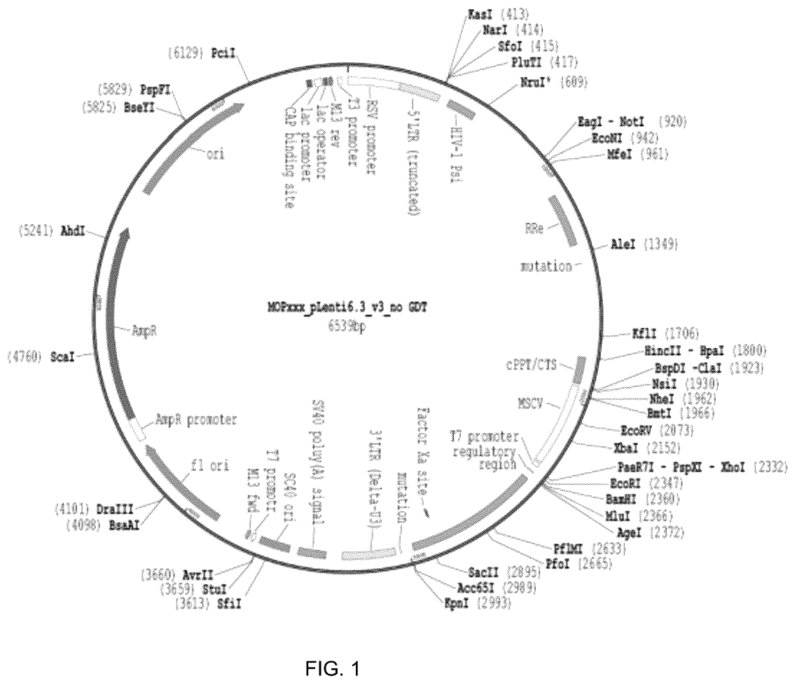

### DETAILED DESCRIPTION

### a. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The terms “nucleic acid”, “nucleic acid molecule”, and “polynucleotide” are used interchangeably herein. The terms “nucleic acid encoding . . . ”, or “nucleic acid molecule encoding . . . ”, should be understood as referring to the sequence of nucleotides which encodes a polypeptide. “Gene” or “coding sequence” or “nucleic acid” or “nucleic” refers to a DNA or RNA region (the transcribed region) which “encodes” a particular polypeptide such as a δT-cell receptor or a γT-cell receptor or a γδTCR or parts thereof. A coding sequence is transcribed (DNA) and translated (RNA) into a polypeptide when placed under the control of an appropriate regulatory region, such as a promoter. A gene may comprise several operably linked fragments, such as a promoter, a 5′ leader sequence, an intron, a coding sequence and a 3′ nontranslated sequence, comprising a polyadenylation site or a signal sequence. A chimeric or recombinant gene (such as the one encoding a δTCR or γTCR chain or a γδTCR comprising the polypeptide as identified herein and operably linked to a promoter) is a gene not normally found in nature, such as a gene in which for example the promoter is not associated in nature with part or all of the transcribed DNA region. “Expression of a gene” refers to the process wherein a gene is transcribed into an RNA and/or translated into an active protein.

A polynucleotide described herein may comprise one or more nucleic acids each encoding a polypeptide, all operably linked to (i.e., in a functional relationship with) one or more regulatory sequences, such as a promoter. Such a polynucleotide may alternatively be referred to herein as a “nucleic acid construct” or “construct”.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to a position in a sequence that is 5′ (i.e., nearer the 5′ end of the strand) relative to the fixed position, and downstream refers to the region that is 3′ (i.e., nearer the 3′ end of the strand) relative to the fixed position.

As used herein, a “regulatory sequence” refers to any genetic element that is known to the skilled person to drive or otherwise regulate expression of nucleic acids in a cell. Such sequences include without limitation promoters, transcription terminators, enhancers, repressors, silencers, kozak sequences, polyA sequences, and the like. A regulatory sequence can, for example, be inducible, non-inducible, constitutive, cell-cycle regulated, metabolically regulated, and the like. A regulatory sequence may be a promoter. As used herein, the term “promoter” refers to a nucleic acid fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is preferentially active in specific types of differentiated cells/tissues, such as preferably a T cell. A preferred promoter is the MSCV promoter. Non-limiting examples of suitable promoters include EF1α, MSCV, EF1 alpha-HTLV-1 hybrid promoter, Moloney murine leukemia virus (MoMuLV or MMLV), Gibbon Ape Leukemia virus (GALV), murine mammary tumor virus (MuMTV or MMTV), Rous sarcoma virus (RSV), MHC class II, clotting Factor IX, insulin promoter, PDX1 promoter, CD11, CD4, CD2, gp47 promoter, PGK, Beta-globin, UbC, MND, and derivatives (i.e. variants) thereof. Examples of these promoters are further described in Poletti and Mavilio (2021), Viruses 13:8; 1526, Kuroda et al. (2008), J Gene Med 10(11):1163-1175, Milone et al. (2009), Mol Ther 17:8; 1453-1464, and Klein et al. (2008), J Biomed Biotechnol 683505, all of which are incorporated herein by reference in their entireties.

“Operably linked” is defined herein as a configuration in which a control sequence such as a promoter sequence or regulating sequence is appropriately placed at a position relative to the nucleotide sequence of interest, preferably coding for a δTCR (or a γTCR) chain or a γδTCR comprising the polypeptide as identified such that the promoter or control or regulating sequence directs or affects the transcription and/or production or expression of the nucleotide sequence of interest, preferably encoding a δTCR (or γTCR) chain or a γδTCR comprising the polypeptide as identified in a cell and/or in a subject. For instance, a promoter is operably linked to a coding sequence if the promoter is able to initiate or regulate the transcription or expression of a coding sequence, in which case the coding sequence should be understood as being “under the control of” the promoter.

A “wild type” protein amino acid sequence can refer to a sequence that is naturally occurring and encoded by a germline genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide. An amino acid sequence that is “derived from” a wild type sequence or other amino acid sequence disclosed herein can refer to an amino acid sequence that differs by one or more amino acids compared to the reference amino acid sequence, for example, containing one or more amino acid insertions, deletions, or substitutions as disclosed herein. The terms “derivative,” “variant,” “variations” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants, “variations” and fragments of a polypeptide can comprise one or more amino acid variations (e.g., mutations, insertions, and deletions), truncations, modifications, or combinations thereof compared to a wild type polypeptide. A part or fragment of a polypeptide may correspond to at least 1%, at least 2%, at least 3%, at least 4%, at least 5%, at least 10%, at least 20%, at least 30%, at least 40% of the length of a polypeptide, such as a polypeptide having an amino acid sequence identified by a specific SEQ ID NO., or having at least 50%, or at least 60%, or at least 70%, or at least 80%, or at least 90% of the length (in amino acids) of the polypeptide.

Within the context of the present application, a protein is represented by an amino acid sequence, and correspondingly a nucleic acid molecule or a polynucleotide is represented by a nucleic acid sequence. Identity and similarity between sequences: throughout this application, it should be understood that for each reference to a specific amino acid sequence using a unique sequence identifier (SEQ ID NO.), the sequence may be replaced by: a polypeptide represented by an amino acid sequence comprising a sequence that has at least 60% sequence identity or similarity with the reference amino acid sequence. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length (in amino acids or nucleotides) of two given SEQ ID NOS or based on a portion thereof. A portion of a full-length sequence may be referred to as a fragment, and preferably means at least 50%, 60%, 70%, 80%, 90%, or 100% of the length (in amino acids or nucleotides) of a reference sequence. “Identity” also refers to the degree of sequence relatedness between two amino acid sequences, or between two nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of ths sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined, for example, by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below:

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P |
| formation | and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

An “antigen” is a molecule or molecular structure that an antigen receptor or an antigen-binding protein can recognize (for example, bind to). An antigen can be or can comprise, for example, a peptide, a polypeptide, a carbohydrate, a chemical, a moiety, a non-peptide antigen, a phosphoantigen, a tumor-associated antigen, a neoantigen, a tumor microenvironment antigen, a microbial antigen, a viral antigen, a bacterial antigen, an autoantigen, a glycan-based antigen, a peptide-based antigen, a lipid-based antigen, or any combination thereof. In some aspects, an antigen is capable of inducing an immune response. In some examples, an antigen binds to an antigen receptor or antigen-binding protein, or induces an immune response, when present in a complex e.g., presented by MHC. In some cases, an antigen adopts a certain conformation in order to bind to an antigen receptor or antigen-binding protein, and/or to induce an immune response, e.g., adopts a conformation in response to the presence or absence of one or more metabolites. Antigen can refer to a whole target molecule, a whole complex, a or a fragment of a target molecule or complex that binds to an antigen receptor or an antigen-binding protein. Antigen receptors that recognize antigens include exogenous antigen-recognition receptors disclosed herein and other antigen-recognition receptors, such as endogenous T cell receptors.

A polypeptide comprising a δT-cell (or γT-cell) receptor chain or part thereof which mediates an anti-tumor response as explained herein may be coupled or linked to an agent to form a “conjugate”. The agent may be selected from the group consisting of a diagnostic agent, a therapeutic agent, an anti-cancer agent, a chemical, a nanoparticle, a chemotherapeutic agent or a fluorochrome.

An “expression construct” or “nucleic acid construct” carries a genome that is able to stabilize and remain episomal in a cell. Within the context of the invention, a cell may mean to encompass a cell used to make the construct or a cell wherein the construct will be administered. Alternatively, a construct is capable of integrating into a cell's genome, e.g. through homologous recombination or otherwise. An exemplary expression construct is one wherein a nucleotide sequence encoding a δTCR (or γTCR) chain or part thereof or a γδTCR is operably linked to a promoter as defined herein wherein said promoter is capable of directing expression of said nucleotide sequence (i.e. coding sequence) in a cell. Such a preferred expression construct is said to comprise an expression cassette. An expression cassette as used herein comprises or consists of a nucleotide sequence encoding a δTCR (or γTCR) chain or part thereof or a γδTCR. An expression construct may comprise two expression cassettes to allow the expression of two polypeptides such as a δTCR and a γTCR chain or part thereof. A viral expression construct is an expression construct which is intended to be used in gene therapy. It is designed to comprise part of a viral genome as later defined herein.

Expression constructs disclosed herein could be prepared using recombinant techniques in which nucleotide sequences encoding said δTCR (or γTCR) chain or part thereof or a γδTCR are expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Typically, a nucleic acid or nucleotide sequence encoding a δTCR (or γTCR) chain or a γδTCR is used in an expression construct or expression vector. The phrase “expression vector” generally refers to a nucleotide sequence that is capable of effecting expression of a gene in a host compatible with such sequences. These expression vectors typically include at least suitable promoter sequences and optionally, transcription termination signals. An additional factor necessary or helpful in effecting expression can also be used as described herein. A nucleic acid or DNA or nucleotide sequence encoding a δTCR (or γTCR) chain or a γδTCR is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi or other eukaryotic cell lines.

A “DNA construct” or “nucleic acid construct” prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been defined herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognized by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment can be employed. In most cases, the replication system is only functional in the cell that is used to make the vector (bacterial cell as E. coli). Most plasmids and vectors do not replicate in the cells infected with the vector. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

Vector: A vector may comprise a nucleic acid construct or an expression construct as earlier defined herein. A vector as described herein may be selected from any genetic element known in the art which can facilitate transfer of nucleic acids between cells, such as, but not limited to, plasmids, transposons, cosmids, chromosomes, artificial chromosomes, viruses, virions, and the like. A vector may also be a chemical vector, such as a lipid complex or naked DNA. “Naked DNA” or “naked nucleic acid” refers to a nucleic acid molecule that is not contained in encapsulating means that facilitates delivery of a nucleic acid into the cytoplasm of a target host cell. Naked DNA may be circular or linear (linearized DNA sequence). Optionally, a naked nucleic acid can be associated with standard means used in the art for facilitating its delivery of the nucleic acid to the target host cell, for example to facilitate the transport of the nucleic acid through the cell membrane.

A vector may be a viral vector and/or a gene therapy vector. A viral vector is a vector that comprises an expression construct as defined above.

A gene therapy vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein.

A “transgene” is herein defined as a gene or a nucleic acid molecule (i.e., a molecule encoding a δTCR or a γTCR chain or a γδTCR or a part thereof) that has been newly introduced into a cell, i.e., a gene that may be present but may normally not be expressed or expressed at an insufficient level in a cell. The transgene may comprise sequences that are native to the cell, sequences that naturally do not occur in the cell and it may comprise combinations of both. A transgene may contain sequences coding for a δTCR or a γTCR chain or a γδTCR or parts thereof and comprising the polypeptide as identified and/or additional proteins as earlier identified herein that may be operably linked to appropriate regulatory sequences for expression of the sequences coding for a δTCR or a γTCR chain or a γδTCR or parts thereof. Preferably, the transgene is not integrated into the host cell's genome.

“Transduction” refers to the delivery of a δTCR chain or a γTCR chain or parts thereof or a γδTCR or parts thereof into a recipient host cell by a viral vector. For example, transduction of a cell by a retroviral or lentiviral vector of the invention leads to transfer of the genome contained in that vector into the transduced cell. In an aspect, the vector is a lentiviral vector.

“Host cell” refers to the cell into which the DNA delivery takes place, such as the T cells of a donor. T cells of the invention may be named engineered cells as further explained below.

A “genetically modified” or “modified cell” refers to a cell in which the nuclear, organellar or extrachromosomal nucleic acid sequences of a cell has been transformed, modified or transduced using recombinant DNA technology to comprise a heterologous nucleic acid molecule, and is used interchangeably with “engineered cell,” “transformed cell,” and “transduced cell.” In one aspect, a genetically modified cell as disclosed herein expresses a protein encoded by a nucleic acid molecule engineered in such manner to contain an insertion of at least one nucleotide, a deletion of at least one nucleotide, and/or a substitution of at least one nucleotide in a sequence encoding at least one heterologous protein. “Engineered cells” refers herein to cells having been engineered, e.g., by the introduction of an exogenous nucleic acid sequence as defined herein. Such a cell has been genetically modified for example by the introduction of for example one or more mutations, insertions and/or deletions in the endogenous gene and/or insertion of a genetic construct in the genome. The modification may have been introduced using recombinant DNA technology. An engineered cell may refer to a cell in isolation or in culture. Engineered cells may be “transduced cells” wherein the cells have been infected with e.g., a modified virus, for example, a retrovirus may be used but other suitable viruses may also be contemplated such as lentiviruses. Non-viral methods may also be used, such as transfections. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient but may also be a stable transfection wherein cells can be selected that have the gene construct integrated in their genome. In some cases, genetic engineering systems such as CRISPR or Argonaute may be utilized to design engineered cells that express a polypeptide described herein.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems. A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

In an aspect, an “engineered cell” has been transformed, modified or transduced to comprise a heterologous or exogenous nucleic acid molecule (i.e. a δTCR chain or a γTCR chain or parts thereof or a γδTCR or parts thereof). In the application, the wording “engineered cell” may be replaced by “modified cell” or “transformed cell” or “transduced cell”. In an aspect, said cell expresses a protein encoded by said nucleic acid molecule. In an aspect, said cell is a TEG.

A “TEG” is a T cell engineered, or genetically modified to express a defined γδ TCR as disclosed herein. In a non-limiting example, a TEG can be an alpha-beta T cell that is engineered to express a defined γδ TCR.

The term “heterologous” refers to an entity that is not native to the cell or species of interest.

The term “ovarian tumor” or “ovarian cancer” is used interchangeably. Ovarian cancer is any cancerous growth arising from different parts of the ovary. The most common form of ovarian cancer (≥80%) arises from the outer lining (epithelium) of the ovary. However, the Fallopian tube (epithelium) is also prone to develop into the same kind of cancer as seen in the ovaries. Since the cells of the ovaries and fallopian tubes are so closely related to each other, the terms “ovarian tumor” and “ovarian cancer” encompass cancerous growth arising from the fallopian tubes. Other forms of ovarian cancer can arise from egg cells (i.e., a germ cell tumor). The risk of ovarian cancer increases with age and decreases with pregnancy. Lifetime risk for ovarian cancer has been estimated at about 1.6%, but women with affected first-degree relatives have a higher (^{˜}5%) risk. Women with a mutated BRCA1 or BRCA2 gene carry a risk between 25% and 60% depending on the specific mutation. Ovarian cancer is the fifth leading cause of death from cancer in women and the leading cause of death from gynecological cancer.

“Pharmaceutical composition” means a mixture of substances suitable for administering to an individual that includes a pharmaceutical agent. As used herein a pharmaceutical composition comprises one or more of receptors, vectors, cells disclosed herein compounded with suitable pharmaceuticals carriers or excipients.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

As used herein, the term “patient”, “subject”, or “test subject” refers to any organism to which provided compound or compounds described herein are administered in accordance with the present invention e.g., for experimental, diagnostic, prophylactic, and/or therapeutic purposes. Typical subjects include animals (e.g., mammals such as mice, rats, rabbits, non-human primates, humans, insects, worms etc.). In an aspect, a subject is a human. In some aspects, a subject may be suffering from, and/or susceptible to a disease, disorder, and/or condition (e.g., cancer such as ovarian cancer, cancer of the fallopian tube(s), peritoneal cancer and breast cancer). In some aspects, a patient is a human possessing one or more female reproductive organs. In some aspects, a patient is a human female (i.e., a woman) that has been diagnosed with a gynecological cancer (e.g., cancer such as ovarian cancer, cancer of the fallopian tube(s), peritoneal cancer and breast cancer). As used herein, a “patient population” or “population of subjects” refers to a plurality of patients or subjects.

The term “effective amount” as used herein is defined as the amount of the molecules of the present invention that are necessary to result in the desired physiological change in the cell or tissue to which it is administered. The term “therapeutically effective amount” as used herein is defined as the amount of the molecules of the present invention that achieves a desired effect with respect to cancer. In this context, a “desired effect” is synonymous with “an anti-tumor activity” as earlier defined herein. A skilled artisan readily recognizes that in many cases the molecules may not provide a cure but may provide a partial benefit, such as alleviation or improvement of at least one symptom or parameter. In some embodiments, a physiological change having some benefit is also considered therapeutically beneficial. Thus, in some embodiments, an amount of molecules that provides a physiological change is considered an “effective amount” or a “therapeutically effective amount.”

As used herein, the disclosure of numerical ranges by numerical endpoints includes all numbers encompassed by that range (e.g., “1 to 5” includes but is not limited to 1, 1.25, 1.5, 1.75, 2, 2.3, 2.5, 2.8, 3, 3.1, 3.3, 3.8, 3.9, 4, 4.25, 4.5, 4.75 and 5). Unless otherwise indicated, all numbers used herein to express quantities, amounts, dimensions, measurements, and the like should be understood as encompassing the specific quantities, amounts, dimensions, measurements and so on, and also as encompassing such instances modified by the term “about.” Accordingly, unless indicated to the contrary, the numerical descriptions set forth herein may vary while remaining well within the teachings of the present disclosure. At the very least, each numerical value should be construed in view of the number of significant digits and by applying routine rounding techniques. As various changes could be made in the above-described cells and methods without departing from the scope of the invention, it is intended that all matter contained in the above description and in the examples given below, shall be interpreted as illustrative and not in a limiting sense. As various changes could be made in the above-described cells and methods without departing from the scope of the invention, it is intended that all matter contained in the above description and in the examples given below, shall be interpreted as illustrative and not in a limiting sense.

### b. Compositions

The present disclosure is based in part on the surprising discovery that γ9δ2T-cell receptors disclosed herein are effective immunotherapy agents against a range of ovarian cancers. The present disclosure encompasses compositions and methods for treating ovarian cancer using the γ9δ2T-cell receptors disclosed herein.

δT-Cell and γT-Cell Receptor Chains or a Part Thereof

Provided in certain aspects described herein are polypeptides comprising a δT-cell receptor chain or a variant or a part or a fragment thereof. In a further aspect the invention provides a δT-cell receptor chain or a part thereof, comprising a CDR3 region, and which δT-cell receptor chain or part thereof is represented by an amino acid sequence as defined herein. Each of these δT-cell receptor chain or part thereof is represented by an amino acid sequence that could be identified using a SEQ ID NO. In an aspect, a δT-cell receptor chain is a δ2T-cell receptor chain.

Provided in certain aspects described herein are polypeptides comprising a γT-cell receptor chain or a variant or part or fragment thereof. In a further aspect the invention provides a γT-cell receptor chain or a part thereof, comprising a CDR3 region, and which γT-cell receptor chain or part thereof is represented by an amino acid sequence as defined herein. Each of these γT-cell receptor chain or part thereof is represented by an amino acid sequence that could be identified using a SEQ ID NO. In an aspect, a γT-cell receptor chain is a γ9T-cell receptor chain.

Part or fragment thereof may mean at least 50% of the length of the SEQ ID NO, or at least 60%, or at least 70%, or at least 80%, or at least 90%. A part or fragment of a polypeptide is preferably a functional part or fragment thereof. It may mean that this part or fragment exhibits a similar activity as the original polypeptide it derives from. In the context of the invention, an activity may be an anti-tumour response as explained later herein. A similar anti-tumour response may mean at least 50% of said anti-tumour response, or at least 60%, or at least 70%, or at least 80%, or at least 90%, or at least 100% or at least 110% or at least 120% or more.

Each δT-cell receptor chain or part thereof comprising a CDR3 region identified herein may also be represented by its coding nucleic acid sequence instead of its amino acid sequence. Therefore, the invention also relates to a nucleic acid molecule encoding said receptor chain or part thereof. The same holds for each of the γT-cell receptor chain or part thereof comprising a CDR3 region identified herein. The same also holds for the TCR identified herein: it can be identified by the receptor chains it comprises or by the nucleic acid molecules encoding the chains it comprises. The same also holds for the T cell expressing said TCR: the T cell can be defined by reference to the receptor chains or parts thereof it expresses or by the nucleic acid molecules encoding these chains or parts thereof it comprises.

In a first aspect, there is provided a δT-cell receptor chain or part thereof comprising a CDR3 region, said δT-cell receptor chain or part thereof being represented by an amino acid sequence, said amino acid sequence comprising at least 60% sequence identity or similarity with one or more of amino acid sequence SEQ ID NOS: 1-3, 7-12, 25-27. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a δT-cell receptor chain or part thereof comprising a CDR3 region, said δT-cell receptor chain or part thereof being represented by an amino acid sequence, said amino acid sequence comprising at least 60% sequence identity or similarity with any one of amino acid sequence SEQ ID NOS: 31-33. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a nucleic acid molecule encoding a δT-cell receptor comprising a nucleotide sequence that has at least 60% sequence identity with one or more of SEQ ID NOS: 4-6 or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a δT-cell receptor chain or part thereof comprising a CDR3 region, said δT-cell receptor chain or part thereof being represented by an amino acid sequence, said amino acid sequence comprising at least 60% sequence identity or similarity with any one of amino acid sequence SEQ ID NO: 7-12. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a γT-cell receptor chain or part thereof comprising a CDR3 region, said γT-cell receptor chain or part thereof being represented by an amino acid sequence, said amino acid sequence comprising at least 80% sequence identity or similarity with any one of amino acid sequence SEQ ID NOS: 13-15, 19-22, 24, 28-30, 34-36. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a γT-cell receptor chain or part thereof comprising a CDR3 region, said γT-cell receptor chain or part thereof being represented by an amino acid sequence, said amino acid sequence comprising at least 80% sequence identity or similarity with any one of amino acid sequence SEQ ID NOS: 13-15, 19-22, 28-30. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a γT-cell receptor chain or part thereof comprising a CDR3 region, said γT-cell receptor chain or part thereof being represented by an amino acid sequence, said amino acid sequence comprising at least 80% sequence identity or similarity with any one of amino acid sequence SEQ ID NOS: 34-36. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a nucleic acid molecule encoding a γT-cell receptor comprising a nucleotide sequence that has at least 60% sequence identity with one or more of SEQ ID NO: 16-18 or fragments thereof. In some aspect, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a γT-cell receptor chain or part thereof comprising a CDR3 region, said γT-cell receptor chain or part thereof being represented by an amino acid sequence, said amino acid sequence comprising at least 60% sequence identity or similarity with any one of amino acid sequence SEQ ID NOS: 19-22. In some aspect, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an aspect, there is provided a T-cell receptor represented by an amino acid sequence, said amino acid sequence comprising at least 60% sequence identity or similarity with one or more of amino acid sequence SEQ ID NOS: 1-3, 7-12, 23, 25-27, 31-33 and/or a T-cell receptor represented by an amino acid sequence, said amino acid sequence comprising at least 60% sequence identity or similarity with one or more of amino acid sequence SEQ ID NOS: 13-15, 19-22, 24, 28-30, 34-36.

In an aspect, there is provided a nucleic acid molecule encoding a δT-cell receptor represented by a nucleotide sequence comprising a nucleotide sequence that has at least 60% sequence identity with SEQ ID NOS: 4-6 or fragments thereof and/or nucleic acid molecule encoding a γT-cell receptor represented by a nucleotide sequence comprising a nucleotide sequence that has at least 60% sequence identity with SEQ ID NOS: 16-18 or fragments thereof. In some aspects, the identity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

TABLE A
Receptor chain sequences, full and partial length as used herein.

| SEQ | | | |
|---|---|---|---|
| ID | | | Seq. |
| NO. | Description | Sequence | Type |
| 1 | δ2-T-cell receptor | MERISSLIHLSLFWAGVMSAIELVPEHQTVPVSIGVPATL | AA |
| | chain (G115)- | RCSMKGEAIGNYYINWYRKTQGNTMTFIYREKDIYGPGFK | |
| | variable domain | DNFQGDIDIAKNLAVLKILAPSERDEGSYYCACDTLGMGG | |
| | | EYTDKLIFGKGTRVTVEPR | |
| 2 | δ2-T-cell receptor | MERISSLIHLSLFWAGVMSAIELVPEHQTVPVSIGVPATL | AA |
| | chain (CL3)- | RCSMKGEAIGNYYINWYRKTQGNTMTFIYREKDIYGPGFK | |
| | variable domain | DNFQGDIDIAKNLAVLKILAPSERDEGSYYCACDLLGYTD | |
| | | KLIFGKGTRVTVEPR | |
| 3 | δ2-T-cell receptor | MERISSLIHLSLFWAGVMSAIELVPEHQTVPVSIGVPATL | AA |
| | chain (CL5)- | RCSMKGEAIGNYYINWYRKTQGNTMTFIYREKDIYGPGFK | |
| | variable domain | DNFQGDIDIAKNLAVLKILAPSERDEGSYYCACDALKRTD | |
| | | TDKLIFGKGTRVTVEPR | |
| 4 | δ2-T-cell receptor | atggagcggatcagcagcctgatccacctgagcctgttct | DNA |
| | chain (G115) | gggccggagtgatgagcgccatcgagctggtgcccgagca | |
| | | ccagaccgtgcccgtgagcatcggcgtgcccgccaccctg | |
| | | cggtgcagcatgaagggcgaggccatcggcaactactaca | |
| | | tcaactggtacagaaagacccagggcaacaccatgacctt | |
| | | catctaccgggagaaggacatctacggccctggcttcaag | |
| | | gacaacttccagggcgacatcgacatcgccaagaacctgg | |
| | | ccgtgctgaagatcctggcccccagcgagagggacgaggg | |
| | | cagctac | |
| 5 | δ2-T-cell receptor | tactgcgcctgcgacaccctgggcatgggcggcgagtaca | DNA |
| | chain (CL3) | ccgacaagctgatcttcggcaagggcacccgggtgaccgt | |
| | | ggagcccagaagccagccccacaccaagcccagcgtgttc | |
| | | gtgatgaagaacggcaccaacgtggcctgcctggtgaaag | |
| | | agttctaccccaaggacatccggatcaacctggtgtccag | |
| | | caagaagatcaccgagttcgaccccgccatcgtgatcagc | |
| | | cccagcggcaagtacaacgccgtgaagctgggcaagtacg | |
| | | aggacagcaacagcgtgacctgcagcgtgcagcacgacaa | |
| | | caagaccgtgcacagcaccgacttcgaggtgaaaaccgac | |
| | | tccaccgaccacgtgaagcccaaagagaccgagaacacca | |
| | | agcagcccagcaagagctgccacaagcccaaggccatcgt | |
| | | gcacaccgagaaggtgaacatgatgagcctgaccgtgctg | |
| | | ggcctgcggatgctgttcgccaagacagtggccgtgaact | |
| | | tcctgctgaccgccaagctgttcttcctgtgaatggagcg | |
| | | gatcagcagcctgatccacctgagcctgttctgggccgga | |
| | | gtgatgagcgccatcgagctggtgcccgagcaccagaccg | |
| | | tgcccgtgagcatcggcgtgcccgccaccctgcggtgcag | |
| | | catgaagggcgaggccatcggcaactactacatcaactgg | |
| | | tacagaaagacccagggcaacaccatgaccttcatctacc | |
| | | gggagaaggacatctacggccctggcttcaaggacaactt | |
| | | ccagggcgacatcgacatcgccaagaacctggccgtgctg | |
| | | aagatcctggcccccagcgagagggacgagggcagctact | |
| | | actgcgcctgcgacctgctgggctacaccgacaagctgat | |
| | | cttcggcaagggcacccgggtgaccgtggagcccagaagc | |
| | | cagccccacaccaagcccagcgtgttcgtgatgaagaacg | |
| | | gcaccaacgtggcctgcctggtgaaagagttctaccccaa | |
| | | ggacatccggatcaacctggtgtccagcaagaagatcacc | |
| | | gagttcgaccccgccatcgtgatcagccccagcggcaagt | |
| | | acaacgccgtgaagctgggcaagtacgaggacagcaacag | |
| | | cgtgacctgcagcgtgcagcacgacaacaagaccgtgcac | |
| | | agcaccgacttcgaggtgaaaaccgactccaccgaccacg | |
| | | tgaagcccaaagagaccgagaacaccaagcagcccagcaa | |
| | | gagctgccacaagcccaaggccatcgtgcacaccgagaag | |
| | | gtgaacatgatgagcctgaccgtgctgggcctgcggatgc | |
| | | tgttcgccaagacagtggccgtgaacttcctgctgaccgc | |
| | | caagctgttcttcctgtga | |
| 6 | δ2-T-cell receptor | atggagcggatcagcagcctgatccacctgagcctgttct | DNA |
| | chain (CL5) | gggccggagtgatgagcgccatcgagctggtgcccgagca | |
| | | ccagaccgtgcccgtgagcatcggcgtgcccgccaccctg | |
| | | cggtgcagcatgaagggcgaggccatcggcaactactaca | |
| | | tcaactggtacagaaagacccagggcaacaccatgacctt | |
| | | catctaccgggagaaggacatctacggccctggcttcaag | |
| | | gacaacttccagggcgacatcgacatcgccaagaacctgg | |
| | | ccgtgctgaagatcctggcccccagcgagagggacgaggg | |
| | | cagctactactgcgcctgcgacgccctgaagagaaccgac | |
| | | accgacaagctgatcttcggcaagggcacccgggtgaccg | |
| | | tggagcccagaagccagccccacaccaagcccagcgtgtt | |
| | | cgtgatgaagaacggcaccaacgtggcctgcctggtgaaa | |
| | | gagttctaccccaaggacatccggatcaacctggtgtcca | |
| | | gcaagaagatcaccgagttcgaccccgccatcgtgatcag | |
| | | ccccagcggcaagtacaacgccgtgaagctgggcaagtac | |
| | | gaggacagcaacagcgtgacctgcagcgtgcagcacgaca | |
| | | acaagaccgtgcacagcaccgacttcgaggtgaaaaccga | |
| | | ctccaccgaccacgtgaagcccaaagagaccgagaacacc | |
| | | aagcagcccagcaagagctgccacaagcccaaggccatcg | |
| | | tgcacaccgagaaggtgaacatgatgagcctgaccgtgct | |
| | | gggcctgcggatgctgttcgccaagacagtggccgtgaac | |
| | | ttcctgctgaccgccaagctgttcttcctgtga | |
| 7 | δ2-T-cell receptor | TLGMGGEY | AA |
| | chain (G115) | | |
| | CDR3 | | |
| 8 | δ2-CDR3 (CL3)- | LLGY | AA |
| | sub | | |
| 9 | δ2-CDR3 (CL5)- | ALKRTD | AA |
| | sub | | |
| 10 | δ2-CDR3 (115) | ACDTLGMGGEYTDKLI | AA |
| 11 | δ2-CDR3 (CL3) | ACDLLGYTDKLI | AA |
| 12 | δ2-CDR3 (CL5) | ACDALKRTDTDKLI | AA |
| 13 | γ9-T-cell receptor | MVSLLHASTLAVLGALCVYGAGHLEQPQISSTKTLSKTAR | AA |
| | chain (G115) | LECVVSGITISATSVYWYRERPGEVIQFLVSISYDGTVRK | |
| | variable domain | ESGIPSGKFEVDRIPETSTSTLTIHNVEKQDIATYYCALW | |
| | | EAQQELGKKIKVFGPGTKLIIT | |
| 14 | γ9-T-cell receptor | MVSLLHASTLAVLGALCVYGAGHLEQPQISSTKTLSKTAR | AA |
| | chain (CL3) | LECVVSGITISATSVYWYRERPGEVIQFLVSISYDGTVRK | |
| | variable domain | ESGIPSGKFEVDRIPETSTSTLTIHNVEKQDIATYYCALW | |
| | | EEELGKKIKVFGPGTKLIIT | |
| 15 | γ9-T-cell receptor | MVSLLHASTLAVLGALCVYGAGHLEQPQISSTKTLSKTAR | AA |
| | chain (CL5) | LECVVSGITISATSVYWYRERPGEVIQFLVSISYDGTVRK | |
| | variable domain | ESGIPSGKFEVDRIPETSTSTLTIHNVEKQDIATYYCALW | |
| | | EIQELGKKIKVFGPGTKLIIT | |
| 16 | γ9-T-cell receptor | atggtgtccctgctgcacgccagcaccctggccgtgctgg | DNA |
| | chain (G115)-full | gcgccctgtgcgtgtatggcgccggacacctggaacagcc | |
| | length | ccagatcagcagcaccaagaccctgagcaagaccgccagg | |
| | | ctggaatgcgtggtgtccggcatcaccatcagcgccacct | |
| | | ccgtgtactggtacagagagagacccggcgaggtcatcca | |
| | | gttcctggtgtccatcagctacgacggcaccgtgcggaaa | |
| | | gagagcggcatccccagcggcaagttcgaggtggacagaa | |
| | | tccccgagaccagcacctccaccctgaccatccacaacgt | |
| | | ggagaagcaggacatcgccacctactactgcgccctgtgg | |
| | | gaggcccagcaggaactgggcaagaaaatcaaggtgttcg | |
| | | gccctggcaccaagctgatcatcaccgacaagcagctgga | |
| | | cgccgacgtgagccccaagcctaccatcttcctgcccagc | |
| | | atcgccgagaccaagctgcagaaggccggcacctacctgt | |
| | | gcctgctggaaaagttcttccccgacgtgatcaagatcca | |
| | | ctgggaggaaaagaagagcaacaccatcctgggcagccag | |
| | | gaaggcaataccatgaaaaccaacgacacctacatgaagt | |
| | | tcagctggctgaccgtgcccgagaagagcctggacaaaga | |
| | | gcacagatgcatcgtccggcacgagaacaacaagaacggc | |
| | | gtggaccaggaaatcatcttcccccccatcaagaccgatg | |
| | | tgatcacaatggaccccaaggacaactgcagcaaggacgc | |
| | | caacgataccctgctgctgcagctgaccaacaccagcgcc | |
| | | tactacatgtatctcctgctgctgctgaagagcgtggtgt | |
| | | acttcgccatcatcacctgctgtctgctgcggcggaccgc | |
| | | cttctgctgcaacggcgagaagagctga | |
| 17 | γ9-T-cell receptor | atggtgtccctgctgcacgccagcaccctggccgtgctgg | DNA |
| | chain (CL3)-full | gcgccctgtgcgtgtatggcgccggacacctggaacagcc | |
| | length | ccagatcagcagcaccaagaccctgagcaagaccgccagg | |
| | | ctggaatgcgtggtgtccggcatcaccatcagcgccacct | |
| | | ccgtgtactggtacagagagagacccggcgaggtcatcca | |
| | | gttcctggtgtccatcagctacgacggcaccgtgcggaaa | |
| | | gagagcggcatccccagcggcaagttcgaggtggacagaa | |
| | | tccccgagaccagcacctccaccctgaccatccacaacgt | |
| | | ggagaagcaggacatcgccacctactactgcgccctgtgg | |
| | | gaggaggaactgggcaagaaaatcaaggtgttcggccctg | |
| | | gcaccaagctgatcatcaccgacaagcagctggacgccga | |
| | | cgtgagccccaagcctaccatcttcctgcccagcatcgcc | |
| | | gagaccaagctgcagaaggccggcacctacctgtgcctgc | |
| | | tggaaaagttcttccccgacgtgatcaagatccactggga | |
| | | ggaaaagaagagcaacaccatcctgggcagccaggaaggc | |
| | | aataccatgaaaaccaacgacacctacatgaagttcagct | |
| | | ggctgaccgtgcccgagaagagcctggacaaagagcacag | |
| | | atgcatcgtccggcacgagaacaacaagaacggcgtggac | |
| | | caggaaatcatcttcccccccatcaagaccgatgtgatca | |
| | | caatggaccccaaggacaactgcagcaaggacgccaacga | |
| | | taccctgctgctgcagctgaccaacaccagcgcctactac | |
| | | atgtatctcctgctgctgctgaagagcgtggtgtacttcg | |
| | | ccatcatcacctgctgtctgctgcggcggaccgccttctg | |
| | | ctgcaacggcgagaagagctgag | |
| 18 | γ9-T-cell receptor | atggtgtccctgctgcacgccagcaccctggccgtgctgg | DNA |
| | chain (CL5)-full | gcgccctgtgcgtgtatggcgccggacacctggaacagcc | |
| | length | ccagatcagcagcaccaagaccctgagcaagaccgccagg | |
| | | ctggaatgcgtggtgtccggcatcaccatcagcgccacct | |
| | | ccgtgtactggtacagagagagacccggcgaggtcatcca | |
| | | gttcctggtgtccatcagctacgacggcaccgtgcggaaa | |
| | | gagagcggcatccccagcggcaagttcgaggtggacagaa | |
| | | tccccgagaccagcacctccaccctgaccatccacaacgt | |
| | | ggagaagcaggacatcgccacctactactgcgccctgtgg | |
| | | gagatccaggaactgggcaagaaaatcaaggtgttcggcc | |
| | | ctggcaccaagctgatcatcaccgacaagcagctggacgc | |
| | | cgacgtgagccccaagcctaccatcttcctgcccagcatc | |
| | | gccgagaccaagctgcagaaggccggcacctacctgtgcc | |
| | | tgctggaaaagttcttccccgacgtgatcaagatccactg | |
| | | ggaggaaaagaagagcaacaccatcctgggcagccaggaa | |
| | | ggcaataccatgaaaaccaacgacacctacatgaagttca | |
| | | gctggctgaccgtgcccgagaagagcctggacaaagagca | |
| | | cagatgcatcgtccggcacgagaacaacaagaacggcgtg | |
| | | gaccaggaaatcatcttcccccccatcaagaccgatgtga | |
| | | tcacaatggaccccaaggacaactgcagcaaggacgccaa | |
| | | cgataccctgctgctgcagctgaccaacaccagcgcctac | |
| | | tacatgtatctcctgctgctgctgaagagcgtggtgtact | |
| | | tcgccatcatcacctgctgtctgctgcggcggaccgcctt | |
| | | ctgctgcaacggcgagaagagc | |
| 19 | γ9-T-cell receptor | AQQ | AA |
| | chain (G115) | | |
| | CDR3 | | |
| 20 | γ9-CDR3 (CL5)- | IQ | AA |
| | sub | | |
| 21 | γ9-CDR3 (115) | ALWEAQQELGKKIKV | AA |
| 22 | γ9-CDR3 (CL5) | ALWEIQELGKKIKV | AA |
| 23 | δ constant domain | SQPHTKPSVFVMKNGTNVACLVKEFYPKDIRINLVSSKKI | AA |
| | TRCD | TEFDPAIVISPSGKYNAVKLGKYEDSNSVTCSVQHDNKTV | |
| | | HSTDFEVKTDSTDHVKPKETENTKQPSKSCHKPKAIVHTE | |
| | | KVNMMSLTVLGLRMLFAKTVAVNFLLTAKLFFL | |
| 24 | γ constant 1 | DKQLDADVSPKPTIFLPSIAETKLQKAGTYLCLLEKFFPD | AA |
| | domain | VIKIHWEEKKSNTILGSQEGNTMKTNDTYMKFSWLTVPEK | |
| | | SLDKEHRCIVRHENNKNGVDQEIIFPPIKTDVITMDPKDN | |
| | | CSKDANDTLLLQLTNTSAYYMYLLLLLKSVVYFAIITCCL | |
| | | LRRTAFCCNGEKS | |
| 25 | δ2-T-cell receptor | SAIELVPEHQTVPVSIGVPATLRCSMKGEAIGNYYINWYR | AA |
| | chain (G115) | KTQGNTMTFIYREKDIYGPGFKDNFQGDIDIAKNLAVLKI | |
| | variable domain | LAPSERDEGSYYCACDTLGMGGEYTDKLIFGKGTRVTVEP | |
| | without signal | R | |
| | peptide | | |
| 26 | δ2-T-cell receptor | SAIELVPEHQTVPVSIGVPATLRCSMKGEAIGNYYINWYR | AA |
| | chain (CL3) | KTQGNTMTFIYREKDIYGPGFKDNFQGDIDIAKNLAVLKI | |
| | variable domain | LAPSERDEGSYYCACDLLGYTDKLIFGKGTRVTVEPR | |
| | without signal\| | | |
| | peptide | | |
| 27 | δ2-T-cell receptor | SAIELVPEHQTVPVSIGVPATLRCSMKGEAIGNYYINWYR | AA |
| | chain (CL5) | KTQGNTMTFIYREKDIYGPGFKDNFQGDIDIAKNLAVLKI | |
| | variable domain | LAPSERDEGSYYCACDALKRTDTDKLIFGKGTRVTVEPR | |
| | without signal\| | | |
| | peptide | | |
| 28 | γ9-T-cell receptor | GAGHLEQPQISSTKTLSKTARLECVVSGITISATSVYWYR | AA |
| | chain (G115) | ERPGEVIQFLVSISYDGTVRKESGIPSGKFEVDRIPETST | |
| | variable domain | STLTIHNVEKQDIATYYCALWEAQQELGKKIKVFGPGTKL | |
| | signal | IIT | |
| | without signal | | |
| | peptide | | |
| 29 | γ9-T-cell receptor | GAGHLEQPQISSTKTLSKTARLECVVSGITISATSVYWYR | AA |
| | chain (G115) | ERPGEVIQFLVSISYDGTVRKESGIPSGKFEVDRIPETST | |
| | variable domain | STLTIHNVEKQDIATYYCALWEEELGKKIKVFGPGTKLII | |
| | without signal | T | |
| | peptide | | |
| 30 | γ9-T-cell receptor | GAGHLEQPQISSTKTLSKTARLECVVSGITISATSVYWYR | AA |
| | chain (CL3) | ERPGEVIQFLVSISYDGTVRKESGIPSGKFEVDRIPETST | |
| | variable domain | STLTIHNVEKQDIATYYCALWEIQELGKKIKVFGPGTKLI | |
| | without signal | IT | |
| | peptide | | |
| 31 | δ2-T-cell receptor | MERISSLIHLSLFWAGVMSAIELVPEHQTVPVSIGVPATL | AA |
| | chain (G115)-full | RCSMKGEAIGNYYINWYRKTQGNTMTFIYREKDIYGPGFK | |
| | length | DNFQGDIDIAKNLAVLKILAPSERDEGSYYCACDTLGMGG | |
| | | EYTDKLIFGKGTRVTVEPRSQPHTKPSVFVMKNGTNVACL | |
| | | VKEFYPKDIRINLVSSKKITEFDPAIVISPSGKYNAVKLG | |
| | | KYEDSNSVTCSVQHDNKTVHSTDFEVKTDSTDHVKPKETE | |
| | | NTKQPSKSCHKPKAIVHTEKVNMMSLTVLGLRMLFAKTVA | |
| | | VNFLLTAKLFFL | |
| 32 | δ2-T-cell receptor | MERISSLIHLSLFWAGVMSAIELVPEHQTVPVSIGVPATL | AA |
| | chain (CL3)-full | RCSMKGEAIGNYYINWYRKTQGNTMTFIYREKDIYGPGFK | |
| | length | DNFQGDIDIAKNLAVLKILAPSERDEGSYYCACDLLGYTD | |
| | | KLIFGKGTRVTVEPRSQPHTKPSVFVMKNGTNVACLVKEF | |
| | | YPKDIRINLVSSKKITEFDPAIVISPSGKYNAVKLGKYED | |
| | | SNSVTCSVQHDNKTVHSTDFEVKTDSTDHVKPKETENTKQ | |
| | | PSKSCHKPKAIVHTEKVNMMSLTVLGLRMLFAKTVAVNFL | |
| | | LTAKLFFL | |
| 33 | δ2-T-cell receptor | MERISSLIHLSLFWAGVMSAIELVPEHQTVPVSIGVPATL | AA |
| | chain (CL5)-full | RCSMKGEAIGNYYINWYRKTQGNTMTFIYREKDIYGPGFK | |
| | length | DNFQGDIDIAKNLAVLKILAPSERDEGSYYCACDALKRTD | |
| | | TDKLIFGKGTRVTVEPRSQPHTKPSVFVMKNGTNVACLVK | |
| | | EFYPKDIRINLVSSKKITEFDPAIVISPSGKYNAVKLGKY | |
| | | EDSNSVTCSVQHDNKTVHSTDFEVKTDSTDHVKPKETENT | |
| | | KQPSKSCHKPKAIVHTEKVNMMSLTVLGLRMLFAKTVAVN | |
| | | FLLTAKLFFL | |
| 34 | γ9-T-cell receptor | MVSLLHASTLAVLGALCVYGAGHLEQPQISSTKTLSKTAR | AA |
| | chain (G115)-full | LECVVSGITISATSVYWYRERPGEVIQFLVSISYDGTVRK | |
| | length | ESGIPSGKFEVDRIPETSTSTLTIHNVEKQDIATYYCALW | |
| | | EAQQELGKKIKVFGPGTKLIITDKQLDADVSPKPTIFLPS | |
| | | IAETKLQKAGTYLCLLEKFFPDVIKIHWEEKKSNTILGSQ | |
| | | EGNTMKTNDTYMKFSWLTVPEKSLDKEHRCIVRHENNKNG | |
| | | VDQEIIFPPIKTDVITMDPKDNCSKDANDTLLLQLTNTSA | |
| | | YYMYLLLLLKSVVYFAIITCCLLRRTAFCCNGEKS | |
| 35 | γ9-T-cell receptor | MVSLLHASTLAVLGALCVYGAGHLEQPQISSTKTLSKTAR | AA |
| | chain (CL3)-full | LECVVSGITISATSVYWYRERPGEVIQFLVSISYDGTVRK | |
| | length | ESGIPSGKFEVDRIPETSTSTLTIHNVEKQDIATYYCALW | |
| | | EEELGKKIKVFGPGTKLIITDKQLDADVSPKPTIFLPSIA | |
| | | ETKLQKAGTYLCLLEKFFPDVIKIHWEEKKSN | |
| | | TILGSQEGNTMKTNDTYMKFSWLTVPEKSLDKEHRCIVRH | |
| | | ENNKNGVDQEIIFPPIKTDVITMDPKDNCSKDANDTLLLQ | |
| | | LTNTSAYYMYLLLLLKSVVYFAIITCCLLRRTAFCCNGEK | |
| | | S | |
| 36 | γ9-T-cell receptor | MVSLLHASTLAVLGALCVYGAGHLEQPQISSTKTLSKTAR | AA |
| | chain (CL5)-full | LECVVSGITISATSVYWYRERPGEVIQFLVSISYDGTVRK | |
| | length | ESGIPSGKFEVDRIPETSTSTLTIHNVEKQDIATYYCALW | |
| | | EIQELGKKIKVFGPGTKLIITDKQLDADVSPKPTIFLPSI | |
| | | AETKLQKAGTYLCLLEKFFPDVIKIHWEEKKSNTILGSQE | |
| | | GNTMKTNDTYMKFSWLTVPEKSLDKEHRCIVRHENNKNGV | |
| | | DQEIIFPPIKTDVITMDPKDNCSKDANDTLLLQLTNTSAY | |
| | | YMYLLLLLKSVVYFAIITCCLLRRTAFCCNGEKS | |

In some aspects, combinations of amino acid sequences of γ9-CDR3 and δ2-CDR3 regions as described above may be combined, for example as listed below in Table B. Non-limiting, exemplary γ9δ2T-cell receptor combination sequences are listed in Table C. In an aspect, there is provided a T-cell receptor represented by an amino acid sequence, said amino acid sequence comprising at least 60% sequence identity or similarity with an amino acid sequence SEQ ID NOS: 37-40. In some aspect, the identity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83% 84%, 85%, 86%, 87%, 88%, 89%, 90%, 91%, 92%, 93%, 94%, 95%, 96%, 97%, 98%, 99% or 100%.

In an aspect, there is provided a nucleic acid molecule encoding a T-cell receptor represented by a nucleotide sequence comprising a nucleotide sequence that has at least 60% sequence identity with SEQ ID NO: 41-44 or fragments thereof. In some aspect, the identity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83% 84%, 85%, 86%, 87%, 88%, 89%, 90%, 91%, 92%, 93%, 94%, 95%, 96%, 97%, 98%, 99% or 100%.

TABLE B
Combinations of γ982T-cell
receptor CDR3 regions

| Combination | γ9-CDR3 | 82-CDR3 |
|---|---|---|
| γ9-cl5/δ2-cl5 | IQ | ALKRTD |
| Y9-G115/δ2-cl5 | AQQ | ALKRTD |
| γ9-cl5/δ2-G115 | IQ | TLGMGGEY |
| Y9-G115/δ2-cl3 | AQQ | LLGY |
| γ9-cl5/δ2-cl5 | ALWEIQELGKKIK | ACDALKRTDTDKLI |
| Y9-G115/δ2-cl5 | ALWEAQQELGKKIKV | ACDALKRTDTDKLI |
| γ9-cl5/δ2-G115 | ALWEIQELGKKIK | ACDTLGMGGEYTDKLI |
| Y9-G115/δ2-cl3 | ALWEAQQELGKKIKV | ACDLLGYTDKLI |

TABLE C
Non-limiting nucleic acid constructs and
encoded combination polypeptide
sequences

| SEQ | | | Type |
|---|---|---|---|
| ID | | | |
| NOS | Combination | Sequence | |
| 37 | γ9-cl5/δ2- | MVSLLHASTLAVLGALCVYGAGHLE | |
| | cl5 | QPQISSTKTLSAAKTARLECVVSGI | |
| | | TISATSVYWYRERPGEVIQFLVSIS | |
| | | YDGTVRKESGIPSGKFEVDRIPETS | |
| | | TSTLTIHNVEKQDIATYYCALWEIQ | |
| | | ELGKKIKVFGPGTKLIITDKQLDAD | |
| | | VSPKPTIFLPSIAETKLQKAGTYLC | |
| | | LLEKFFPDVIKIHWEEKKSNTILGS | |
| | | QEGNTMKINDTYMKFSWLTVPEKSL | |
| | | DKEHRCIVRHENNKNGVDQEIIFPP | |
| | | IKTDVITMDPKDNCSKDANDTLLLQ | |
| | | LTNTSAYYMYLLLLLKSVVYFAIIT | |
| | | CCLLRRTAFCCNGEKSGSGEGRGSL | |
| | | LTCGDVEENPGPMERISSLIHLSLF | |
| | | WAGVMSAIELVPEHQTVPVSIGVPA | |
| | | TLRCSMKGEAIGNYYINWYRKTQGN | |
| | | TMTFIYREKDIYGPGFKDNFQGDID | |
| | | IAKNLAVLKILAPSERDEGSYYCAC | |
| | | DALKRTDTDKLIFGKGTRVTVEPRS | |
| | | QPHTKPSVFVMKNGTNVACLVKEFY | |
| | | PKDIRINLVSSKKITEFDPAIVISP | |
| | | SGKYNAVKLGKYEDSNSVTCSVQHD | |
| | | NKTVHSTDFEVKTDSTDHVKPKETE | |
| | | NTKQPSKSCHKPKAIVHTEKVNMMS | |
| | | LTVLGLRMLFAKTVAVNFLLTAKLF | |
| | | FL | |
| 38 | γ9-G115/ | MVSLLHASTLAVLGALCVYGAGHLE | |
| | δ2-cl5 | QPQISSTKTLSKTARLECVVSGITI | |
| | | SATSVYWYRERPGEVIQFLVSISYD | |
| | | GTVRKESGIPSGKFEVDRIPETSTS | |
| | | TLTIHNVEKQDIATYYCALWEAQQE | |
| | | LGKKIKVFGPGTKLIITDKQLDADV | |
| | | SPKPTIFLPSIAETKLQKAGTYLCL | |
| | | LEKFFPDVIKIHWEEKKSNTILGSQ | |
| | | EGNTMKTNDTYMKFSWLTVPEKSLD | |
| | | KEHRCIVRHENNKNGVDQEIIFPPI | |
| | | KTDVITMDPKDNCSKDANDTLLLQL | |
| | | TNTSAYYMYLLLLLKSVVYFAIITC | |
| | | CLLRRTAFCCNGEKSGSGEGRGSLL | |
| | | TCGDVEENPGPMERISSLIHLSLFW | |
| | | AGVMSAIELVPEHQTVPVSIGVPAT | |
| | | LRCSMKGEAIGNYYINWYRKTQGNT | |
| | | MTFIYREKDIYGPGFKDNFQGDIDI | |
| | | AKNLAVLKILAPSERDEGSYYCACD | |
| | | ALKRTDTDKLIFGKGTRVTVEPRSQ | |
| | | PHTKPSVFVMKNGTNVACLVKEFYP | |
| | | KDIRINLVSSKKITEFDPAIVISPS | |
| | | GKYNAVKLGKYEDSNSVTCSVQHDN | |
| | | KTVHSTDFEVKTDSTDHVKPKETEN | |
| | | TKQPSKSCHKPKAIVHTEKVNMMSL | |
| | | TVLGLRMLFAKTVAVNFLLTAKLFF | |
| | | L | |
| 39 | γ9-cl5/δ2- | MVSLLHASTLAVLGALCVYGAGHLE | |
| | G115 | QPQISSTKTLSKTARLECVVSGITI | |
| | | SATSVYWYRERPGEVIQFLVSISYD | |
| | | GTVRKESGIPSGKFEVDRIPETSTS | |
| | | TLTIHNVEKQDIATYYCALWEIQEL | |
| | | GKKIKVFGPGTKLIITDKQLDADVS | |
| | | PKPTIFLPSIAETKLQKAGTYLCLL | |
| | | EKFFPDVIKIHWEEKKSNTILGSQE | |
| | | GNTMKTNDTYMKFSWLTVPEKSLDK | |
| | | EHRCIVRHENNKNGVDQEIIFPPIK | |
| | | TDVITMDPKDNCSKDANDTLLLQLT | |
| | | NTSAYYMYLLLLLKSVVYFAIITCC | |
| | | LLRRTAFCCNGEKSGSGEGRGSLLT | |
| | | CGDVEENPGPMERISSLIHLSLFWA | |
| | | GVMSAIELVPEHQTVPVSIGVPATL | |
| | | RCSMKGEAIGNYYINWYRKTQGNTM | |
| | | TFIYREKDIYGPGFKDNFQGDIDIA | |
| | | KNLAVLKILAPSERDEGSYYCACDT | |
| | | LGMGGEYTDKLIFGKGTRVTVEPRS | |
| | | QPHTKPSVFVMKNGTNVACLVKEFY | |
| | | PKDIRINLVSSKKITEFDPAIVISP | |
| | | SGKYNAVKLGKYEDSNSVTCSVQHD | |
| | | NKTVHSTDFEVKTDSTDHVKPKETE | |
| | | NTKQPSKSCHKPKAIVHTEKVNMMS | |
| | | LTVLGLRMLFAKTVAVNFLLTAKLF | |
| | | FL | |
| 40 | γ9-G115/ | MVSLLHASTLAVLGALCVYGAGHLE | |
| | δ2-cl3 | QPQISSTKTLSKTARLECVVSGITI | |
| | | SATSVYWYRERPGEVIQFLVSISYD | |
| | | GTVRKESGIPSGKFEVDRIPETSTS | |
| | | TLTIHNVEKQDIATYYCALWEAQQE | |
| | | LGKKIKVFGPGTKLIITDKQLDADV | |
| | | SPKPTIFLPSIAETKLQKAGTYLCL | |
| | | LEKFFPDVIKIHWEEKKSNTILGSQ | |
| | | EGNTMKTNDTYMKFSWLTVPEKSLD | |
| | | KEHRCIVRHENNKNGVDQEIIFPPI | |
| | | KTDVITMDPKDNCSKDANDTLLLQL | |
| | | TNTSAYYMYLLLLLKSVVYFAIITC | |
| | | CLLRRTAFCCNGEKSGSGEGRGSLL | |
| | | TCGDVEENPGPMERISSLIHLSLFW | |
| | | AGVMSAIELVPEHQTVPVSIGVPAT | |
| | | LRCSMKGEAIGNYYINWYRKTQGNT | |
| | | MTFIYREKDIYGPGFKDNFQGDIDI | |
| | | AKNLAVLKILAPSERDEGSYYCACD | |
| | | LLGYTDKLIFGKGTRVTVEPRSQPH | |
| | | TKPSVFVMKNGTNVACLVKEFYPKD | |
| | | IRINLVSSKKITEFDPAIVISPSGK | |
| | | YNAVKLGKYEDSNSVTCSVQHDNKT | |
| | | VHSTDFEVKTDSTDHVKPKETENTK | |
| | | QPSKSCHKPKAIVHTEKVNMMSLTV | |
| | | LGLRMLFAKTVAVNFLLTAKLFFL | |
| 41 | γ9-cl5/ | atggtgtccctgctgcacgccagca | DNA |
| | δ2-cl5 | ccctggccgtgctgggcgccctgtg | |
| | | cgtgtatggcgccggacacctggaa | |
| | | cagccccagatcagcagcaccaaga | |
| | | ccctgagcaagaccgccaggctgga | |
| | | atgcgtggtgtccggcatcaccatc | |
| | | agcgccacctccgtgtactggtaca | |
| | | gagagagacccggcgaggtcatcca | |
| | | gttcctggtgtcctcagctacgacg | |
| | | gcaccgtgcggaaagagagcggcat | |
| | | ccccagcggcaagttcgaggtggac | |
| | | agaatccccgagaccagcacctcca | |
| | | ccctgaccatccacaacgtggagaa | |
| | | gcaggacatcgccacctactactgc | |
| | | gccctgtgggagatccaggaactgg | |
| | | gcaagaaaatcaaggtgttcggccc | |
| | | tggcaccaagctgatcatcaccgac | |
| | | aagcagctggacgccgacgtgagcc | |
| | | ccaagcctaccatcttcctgcccag | |
| | | catcgccgagaccaagctgcagaag | |
| | | gccggcacctacctgtgcctgctgg | |
| | | aaaagttcttccccgacgtgatcaa | |
| | | gatccactgggaggaaaagaagagc | |
| | | aacaccatcctgggcagccaggaag | |
| | | gcaataccatgaaaaccaacgacac | |
| | | ctacatgaagttcagctggctgacc | |
| | | gtgcccgagaagagcctggacaaag | |
| | | agcacagatgcatcgtccggcacga | |
| | | gaacaacaagaacggcgtggaccag | |
| | | gaaatcatcttcccccccatcaaga | |
| | | ccgatgtgatcacaatggaccccaa | |
| | | ggacaactgcagcaaggacgccaac | |
| | | gataccctgctgctgcagctgacca | |
| | | acaccagcgcctactacatgtatct | |
| | | cctgctgctgctgaagagcgtggtg | |
| | | tacttcgccatcatcacctgctgtc | |
| | | tgctgcggcggaccgccttctgctg | |
| | | caacggcgagaagagcggcagcggc | |
| | | gaaggccgcggcagcctgctgacct | |
| | | gcggcgatgtggaagaaaaccctgg | |
| | | cccgatggagcggatcagcagcctg | |
| | | atccacctgagcctgttctgggccg | |
| | | gagtgatgagcgccatcgagctggt | |
| | | gcccgagcaccagaccgtgcccgtg | |
| | | agcatcggcgtgcccgccaccctgc | |
| | | ggtgcagcatgaagggcgaggccat | |
| | | cggcaactactacatcaactggtac | |
| | | agaaagacccagggcaacaccatga | |
| | | ccttcatctaccgggagaaggacat | |
| | | ctacggccctggcttcaaggacaac | |
| | | ttccagggcgacatcgacatcgcca | |
| | | agaacctggccgtgctgaagatcct | |
| | | ggcccccagcgagagggacgagggc | |
| | | agctactactgcgcctgcgacgccc | |
| | | tgaagagaaccgacaccgacaagct | |
| | | gatcttcggcaagggcacccgggtg | |
| | | accgtggagcccagaagccagcccc | |
| | | acaccaagcccagcgtgttcgtgat | |
| | | gaagaacggcaccaacgtggcctgc | |
| | | ctggtgaaagagttctaccccaagg | |
| | | acatccggatcaacctggtgtccag | |
| | | caagaagatcaccgagttcgacccc | |
| | | gccatcgtgatcagccccagcggca | |
| | | agtacaacgccgtgaagctgggcaa | |
| | | gtacgaggacagcaacagcgtgacc | |
| | | tgcagcgtgcagcacgacaacaaga | |
| | | ccgtgcacagcaccgacttcgaggt | |
| | | gaaaaccgactccaccgaccacgtg | |
| | | aagcccaaagagaccgagaacacca | |
| | | agcagcccagcaagagctgccacaa | |
| | | gcccaaggccatcgtgcacaccgag | |
| | | aaggtgaacatgatgagcctgaccg | |
| | | tgctgggcctgcggatgctgttcgc | |
| | | caagacagtggccgtgaacttcctg | |
| | | ctgaccgccaagctgttcttcctgt | |
| | | ga | |
| 42 | γ9-G115/ | atggtgtccctgctgcacgccagca | DNA |
| | δ2-cl5 | ccctggccgtgctgggcgccctgtg | |
| | | cgtgtatggcgccggacacctggaa | |
| | | cagccccagatcagcagcaccaaga | |
| | | ccctgagcaagaccgccaggctgga | |
| | | atgcgtggtgtccggcatcaccatc | |
| | | agcgccacctccgtgtactggtaca | |
| | | gagagagacccggcgaggtcatcca | |
| | | gttcctggtgtccatcJagctacga | |
| | | cggcaccgtgcggaaagagagcggc | |
| | | atccccagcggcaagttcgaggtgg | |
| | | acagaatccccgagaccagcacctc | |
| | | caccctgaccatccacaacgtggag | |
| | | aagcaggacatcgccacctactact | |
| | | gcgccctgtgggaggcccagcagga | |
| | | actgggcaagaaaatcaaggtgttc | |
| | | ggccctggcaccaagctgatcatca | |
| | | ccgacaagcagctggacgccgacgt | |
| | | gagccccaagcctaccatcttcctg | |
| | | cccagcatcgccgagaccaagctgc | |
| | | agaaggccggcacctacctgtgcct | |
| | | gctggaaaagttcttccccgacgtg | |
| | | atcaagatccactgggaggaaaaga | |
| | | agagcaacaccatcctgggcagcca | |
| | | ggaaggcaataccatgaaaaccaac | |
| | | gacacctacatgaagttcagctggc | |
| | | tgaccgtgcccgagaagagcctgga | |
| | | caaagagcacagatgcatcgtccgg | |
| | | cacgagaacaacaagaacggcgtgg | |
| | | accaggaaatcatcttcccccccat | |
| | | caagaccgatgtgatcacaatggac | |
| | | cccaaggacaactgcagcaaggacg | |
| | | ccaacgataccctgctgctgcagct | |
| | | gaccaacaccagcgcctactacatg | |
| | | tatctcctgctgctgctgaagagcg | |
| | | tggtgtacttcgccatcatcacctg | |
| | | ctgtctgctgcggcggaccgccttc | |
| | | tgctgcaacggcgagaagagctgag | |
| | | gcagcggcgaaggccgcggcagcct | |
| | | gctgacctgcggcgatgtggaagaa | |
| | | aaccctggcccgatggagcggatca | |
| | | gcagcctgatccacctgagcctgtt | |
| | | ctgggccggagtgatgagcgccatc | |
| | | gagctggtgcccgagcaccagaccg | |
| | | tgcccgtgagcatcggcgtgcccgc | |
| | | caccctgcggtgcagcatgaagggc | |
| | | gaggccatcggcaactactacatca | |
| | | actggtacagaaagacccagggcaa | |
| | | caccatgaccttcatctaccgggag | |
| | | aaggacatctacggccctggcttca | |
| | | aggacaacttccagggcgacatcga | |
| | | catcgccaagaacctggccgtgctg | |
| | | aagatcctggcccccagcgagaggg | |
| | | acgagggcagctactactgcgcctg | |
| | | cgacgccctgaagagaaccgacacc | |
| | | gacaagctgatcttcggcaagggca | |
| | | cccgggtgaccgtggagcccagaag | |
| | | ccagccccacaccaagcccagcgtg | |
| | | ttcgtgatgaagaacggcaccaacg | |
| | | tggcctgcctggtgaaagagttcta | |
| | | ccccaaggacatccggatcaacctg | |
| | | gtgtccagcaagaagatcaccgagt | |
| | | tcgaccccgccatcgtgatcagccc | |
| | | cagcggcaagtacaacgccgtgaag | |
| | | ctgggcaagtacgaggacagcaaca | |
| | | gcgtgacctgcagcgtgcagcacga | |
| | | caacaagaccgtgcacagcaccgac | |
| | | ttcgaggtgaaaaccgactccaccg | |
| | | accacgtgaagcccaaagagaccga | |
| | | gaacaccaagcagcccagcaagagc | |
| | | tgccacaagcccaaggccatcgtgc | |
| | | acaccgagaaggtgaacatgatgag | |
| | | cctgaccgtgctgggcctgcggatg | |
| | | ctgttcgccaagacagtggccgtga | |
| | | acttcctgctgaccgccaagctgtt | |
| | | cttcctgtga | |
| 43 | γ9-cl5/ | δ2-atggtgtccctgctgcacgcca | DNA |
| | G115 | gcaccctggccgtgctgggcgccct | |
| | | gtgcgtgtatggcgccggacacctg | |
| | | gaacagccccagatcagcagcacca | |
| | | agaccctgagcaagaccgccaggct | |
| | | ggaatgcgtggtgtccggcatcacc | |
| | | atcagcgccacctccgtgtactggt | |
| | | acagagagagacccggcgaggtcat | |
| | | ccagttcctggtgtccatcagctac | |
| | | gacggcaccgtgcggaaagagagcg | |
| | | gcatccccagcggcaagttcgaggt | |
| | | ggacagaatccccgagaccagcacc | |
| | | tccaccctgaccatccacaacgtgg | |
| | | agaagcaggacatcgccacctacta | |
| | | ctgcgccctgtgggagatccaggaa | |
| | | ctgggcaagaaaatcaaggtgttcg | |
| | | gccctggcaccaagctgatcatcac | |
| | | cgacaagcagctggacgccgacgtg | |
| | | agccccaagcctaccatcttcctgc | |
| | | ccagcatcgccgagaccaagctgca | |
| | | gaaggccggcacctacctgtgcctg | |
| | | ctggaaaagttcttccccgacgtga | |
| | | tcaagatccactgggaggaaaagaa | |
| | | gagcaacaccatcctgggcagccag | |
| | | gaaggcaataccatgaaaaccaacg | |
| | | acacctacatgaagttcagctggct | |
| | | gaccgtgcccgagaagagcctggac | |
| | | aaagagcacagatgcatcgtccggc | |
| | | acgagaacaacaagaacggcgtgga | |
| | | ccaggaaatcatcttcccccccatc | |
| | | aagaccgatgtgatcacaatggacc | |
| | | ccaaggacaactgcagcaaggacgc | |
| | | caacgataccctgctgctgcagctg | |
| | | accaacaccagcgcctactacatgt | |
| | | atctcctgctgctgctgaagagcgt | |
| | | ggtgtacttcgccatcatcacctgc | |
| | | tgtctgctgcggcggaccgccttct | |
| | | gctgcaacggcgagaagagcggcag | |
| | | cggcgaaggccgcggcagcctgctg | |
| | | acctgcggcgatgtggaagaaaacc | |
| | | ctggcccgatggagcggatcagcag | |
| | | cctgatccacctgagcctgttctgg | |
| | | gccggagtgatgagcgccatcgagc | |
| | | tggtgcccgagcaccagaccgtgcc | |
| | | cgtgagcatcggcgtgcccgccacc | |
| | | ctgcggtgcagcatgaagggcgagg | |
| | | ccatcggcaactactacatcaactg | |
| | | gtacagaaagacccagggcaacacc | |
| | | atgaccttcatctaccgggagaagg | |
| | | acatctacggccctggcttcaagga | |
| | | caacttccagggcgacatcgacatc | |
| | | gccaagaacctggccgtgctgaaga | |
| | | tcctggcccccagcgagagggacga | |
| | | gggcagctactactgcgcctgcgac | |
| | | accctgggcatgggcggcgagtaca | |
| | | ccgacaagctgatcttcggcaaggg | |
| | | cacccgggtgaccgtggagcccaga | |
| | | agccagccccacaccaagcccagcg | |
| | | tgttcgtgatgaagaacggcaccaa | |
| | | cgtggcctgcctggtgaaagagttc | |
| | | taccccaaggacatccggatcaacc | |
| | | tggtgtccagcaagaagatcaccga | |
| | | gttcgaccccgccatcgtgatcagc | |
| | | cccagcggcaagtacaacgccgtga | |
| | | agctgggcaagtacgaggacagcaa | |
| | | cagcgtgacctgcagcgtgcagcac | |
| | | gacaacaagaccgtgcacagcaccg | |
| | | acttcgaggtgaaaaccgactccac | |
| | | cgaccacgtgaagcccaaagagacc | |
| | | gagaacaccaagcagcccagcaaga | |
| | | gctgccacaagcccaaggccatcgt | |
| | | gcacaccgagaaggtgaacatgatg | |
| | | agcctgaccgtgctgggcctgcgga | |
| | | tgctgttcgccaagacagtggccgt | |
| | | gaacttcctgctgaccgccaagctg | |
| | | ttcttcctgtga | |
| 44 | γ9-G115/δ2- | atggtgtccctgctgcacgccagca | DNA |
| | cl3 | ccctggccgtgctgggcgccctgtg | |
| | | cgtgtatggcgccggacacctggaa | |
| | | cagccccagatcagcagcaccaaga | |
| | | ccctgagcaagaccgccaggctgga | |
| | | atgcgtggtgtccggcatcaccatc | |
| | | agcgccacctccgtgtactggtaca | |
| | | gagagagacccggcgaggtcatcca | |
| | | gttcctggtgtccatcagctacgac | |
| | | ggcaccgtgcggaaagagagcggca | |
| | | tccccagcggcaagttcgaggtgga | |
| | | cagaatccccgagaccagcacctcc | |
| | | accctgaccatccacaacgtggaga | |
| | | agcaggacatcgccacctactactg | |
| | | cgccctgtgggaggcccagcaggaa | |
| | | ctgggcaagaaaatcaaggtgttcg | |
| | | gccctggcaccaagctgatcatcac | |
| | | cgacaagcagctggacgccgacgtg | |
| | | agccccaagcctaccatcttcctgc | |
| | | ccagcatcgccgagaccaagctgca | |
| | | gaaggccggcacctacctgtgcctg | |
| | | ctggaaaagttcttccccgacgtga | |
| | | tcaagatccactgggaggaaaagaa | |
| | | gagcaacaccatcctgggcagccag | |
| | | gaaggcaataccatgaaaaccaacg | |
| | | acacctacatgaagttcagctggct | |
| | | gaccgtgcccgagaagagcctggac | |
| | | aaagagcacagatgcatcgtccggc | |
| | | acgagaacaacaagaacggcgtgga | |
| | | ccaggaaatcatcttcccccccatc | |
| | | aagaccgatgtgatcacaatggacc | |
| | | ccaaggacaactgcagcaaggacgc | |
| | | caacgataccctgctgctgcagctg | |
| | | accaacaccagcgcctactacatgt | |
| | | atctcctgctgctgctgaagagcgt | |
| | | ggtgtacttcgccatcatcacctgc | |
| | | tgtctgctgcggcggaccgccttct | |
| | | gctgcaacggcgagaagagctgagg | |
| | | cagcggcgaaggccgcggcagcctg | |
| | | ctgacctgcggcgatgtggaagaaa | |
| | | accctggcccgatggagcggatcag | |
| | | cagcctgatccacctgagcctgttc | |
| | | tgggccggagtgatgagcgccatcg | |
| | | agctggtgcccgagcaccagaccgt | |
| | | gcccgtgagcatcggcgtgcccgcc | |
| | | accctgcggtgcagcatgaagggcg | |
| | | aggccatcggcaactactacatcaa | |
| | | ctggtacagaaagacccagggcaac | |
| | | accatgaccttcatctaccgggaga | |
| | | aggacatctacggccctggcttcaa | |
| | | ggacaacttccagggcgacatcgac | |
| | | atcgccaagaacctggccgtgctga | |
| | | agatcctggcccccagcgagaggga | |
| | | cgagggcagctactactgcgcctgc | |
| | | gacctgctgggctacaccgacaagc | |
| | | tgatcttcggcaagggcacccgggt | |
| | | gaccgtggagcccagaagccagccc | |
| | | cacaccaagcccagcgtgttcgtga | |
| | | tgaagaacggcaccaacgtggcctg | |
| | | cctggtgaaagagttctaccccaag | |
| | | gacatccggatcaacctggtgtcca | |
| | | gcaagaagatcaccgagttcgaccc | |
| | | cgccatcgtgatcagccccagcggc | |
| | | aagtacaacgccgtgaagctgggca | |
| | | agtacgaggacagcaacagcgtgac | |
| | | ctgcagcgtgcagcacgacaacaag | |
| | | accgtgcacagcaccgacttcgagg | |
| | | tgaaaaccgactccaccgaccacgt | |
| | | gaagcccaaagagaccgagaacacc | |
| | | aagcagcccagcaagagctgccaca | |
| | | agcccaaggccatcgtgcacaccga | |
| | | gaaggtgaacatgatgagcctgacc | |
| | | gtgctgggcctgcggatgctgttcg | |
| | | ccaagacagtggccgtgaacttcct | |
| | | gctgaccgccaagctgttcttcctg | |
| | | tga | |

In some aspects, the nucleic acid constructs provided herein may be operably linked to a suitable promoter to facilitate expression of the encoded T-cell or γT-cell receptor chains in a suitable host cell. Exemplary promoter sequences are provided in Table D.

TABLE D
Promoter sequences

| SEQ ID | | | |
|---|---|---|---|
| NOS | Name | Sequence | Type |
| 45 | MSCV | tgaaagaccccacctgtaggtttgg | DNA |
| | promoter | caagctagcttaagtaacgccattt | |
| | | tgcaaggcatggaaaatacataact | |
| | | gagaatagagaagttcagatcaagg | |
| | | ttaggaacagagagacagcagaata | |
| | | tgggccaaacaggatatctgtggta | |
| | | agcagttcctgccccggctcagggc | |
| | | caagaacagatggtccccagatgcg | |
| | | gtcccgccctcagcagtttctagag | |
| | | aaccatcagatgtttccagggtgcc | |
| | | ccaaggacctgaaaatgaccctgtg | |
| | | ccttatttgaactaaccaatcagtt | |
| | | cgcttctcgcttctgttcgcgcgtt | |
| | | tctgctccccgagctcaataaaaga | |
| | | gcccacaacccctcact | |
| 46 | MMLV | aatgaaagaccccacctgtaggttt | DNA |
| | promoter | ggcaagctagcttaagtaacgccat | |
| | EF1α | tttgcaaggcatggaaaaatacata | |
| | | actgagaatagaaaagttcagatca | |
| | | aggtcaggaacagatggaacagctg | |
| | | aatatgggccaaacaggatatctgt | |
| | | ggtaagcagttcctgccccggctca | |
| | | gggccaagaacagatggaacagctg | |
| | | aatatgggccaaacaggatatctgt | |
| | | ggtaagcagttcctgccccggctca | |
| | | gggccaagaacagatggtccccaga | |
| | | tgcggtccagccctcagcagtttct | |
| | | agagaaccatcagatgtttccaggg | |
| | | tgccccaaggacctgaaatgaccct | |
| | | gtgccttatttgaactaaccaatca | |
| | | gttcgcttctcgcttctgttcgcgc | |
| | | gcttatgctccccgagctcaataaa | |
| | | agagcccacaacccctcactcgggg | |
| | | cgccagtcctccgattgactgagtc | |
| | | gcccgggtacccgtgtatccaataa | |
| | | accctcttgcagttgcatccgactt | |
| | | gtggtctcgctgttccttgggaggg | |
| | | tctcctctgagtgattgactacccg | |
| | | tcagcgggggtctttcatt | |
| | | gctccggtgcccgtcagtgggcaga | DNA |
| 47 | promoter | gcgcacatcgcccacagtccccgag | |
| | | aagttggggggaggggtcggcaatt | |
| | | gaaccggtgcctagagaaggtggcg | |
| | | cggggtaaactgggaaagtgatgtc | |
| | | gtgtactggctccgcctttttcccg | |
| | | agggtgggggagaaccgtatataag | |
| | | tgcagtagtcgccgtgaacgttctt | |
| | | tttcgcaacgggtttgccgccagaa | |
| | | cacaggtaagtgccgtgtgtggttc | |
| | | ccgcgggcctggcctctttacgggt | |
| | | tatggcccttgcgtgccttgaatta | |
| | | cttccacgcccctggctgcagtacg | |
| | | tgattcttgatcccgagcttcgggt | |
| | | tggaagtgggtgggagagttcgagg | |
| | | ccttgcgcttaaggagccccttcgc | |
| | | ctcgtgcttgagttgaggcctggct | |
| | | tgggcgctggggccgccgcgtgcga | |
| | | atctggtggcaccttcgcgcctgtc | |
| | | tcgctgctttcgataagtctctagc | |
| | | catttaaaatttttgatgacctgct | |
| | | gcgacgctttttttctggcaagata | |
| | | gtcttgtaaatgcgggccaagatct | |
| | | gcacactggtatttcggtttttggg | |
| | | gccgcgggcggcgacggggcccgtg | |
| | | cgtcccagcgcacatgttcggcgag | |
| | | gcggggcctgcgagcgcggccaccg | |
| | | agaatcggacgggggtagtctcaag | |
| | | ctggccggcctgctctggtgcctgg | |
| | | cctcgcgccgccgtgtatcgccccg | |
| | | ccctgggcggcaaggctggcccggt | |
| | | cggcaccagttgcgtgagcggaaag | |
| | | atggccgcttcccggccctgctgca | |
| | | gggagctcaaaatggaggacgcggc | |
| | | gctcgggagagcgggcgggtgagtc | |
| | | acccacacaaaggaaaagggccttt | |
| | | ccgtcctcagccgtcgcttcatgtg | |
| | | actccacggagtaccgggcgccgtc | |
| | | caggcacctcgattagttctcgagc | |
| | | ttttggagtacgtcgtctttaggtt | |
| | | ggggggaggggttttatgcgatgga | |
| | | gtttccccacactgagtgggtggag | |
| | | actgaagttaggccagcttggcact | |
| | | tgatgtaattctccttggaatttgc | |
| | | cctttttgagtttggatcttggttc | |
| | | attctcaagcctcagacagtggttc | |
| | | aaagtttttttcttccatttcaggt | |
| | | gtcgtga | |
| 48 | MND | tttatttagtctccagaaaaagggg | DNA |
| | promoter | ggaatgaaagaccccacctgtaggt | |
| | | ttggcaagctaggatcaaggttagg | |
| | | aacagagagacagcagaatatgggc | |
| | | caaacaggatatctgtggtaagcag | |
| | | ttcctgccccggctcagggccaaga | |
| | | acagttggaacagcagaatatgggc | |
| | | caaacaggatatctgtggtaagcag | |
| | | ttcctgccccggctcagggccaaga | |
| | | acagatggtccccagatgcggtccc | |
| | | gccctcagcagtttctagagaacca | |
| | | tcagatgtttccagggtgccccaag | |
| | | gacctgaaatgaccctgtgccttat | |
| | | ttgaactaaccaatcagttcgcttc | |
| | | tcgcttctgttcgcgcgcttctgct | |
| | | ccccgagctcaataaaagagccca | |

Each of the preferred δT-cell or γT-cell receptor chains or parts thereof as described above by sequence identity (defined by reference to an amino acid sequence or by reference to a nucleic acid molecule encoding them) and as encompassed by the invention are in some aspect considered to be able to exhibit an anti-tumor activity/response in particular anti-ovarian tumor response, as detailed further herein.

In an aspect, a variant or part of a δT-cell (or γT-cell) receptor chain described herein is a soluble polypeptide. Such a soluble polypeptide may also be called a binding unit. Such a soluble polypeptide can include various forms to binding entities such as a TCR, antibody, scFv, BCR, V_{H}H, or any combination thereof. In some cases, at least a portion or fragment or part of a TCR, such as Vγ9Vδ2 can be generated and utilized in a composition (preferably a pharmaceutical compositions) as described herein. For example, TCR-antibody chimeras can be generated and tested before arriving at a desired chimera. For example, γδ-variable domains can replace heavy and light chain variable domains of an antibody. In addition to enhanced binding, an Fc domain of an antibody can mediate cytotoxicity through Fcγ-receptor positive immune cells and/or a complementary system. In some cases, TCR-antibody chimeras can be generated using HEK293 cells and subsequently purified using protein A affinity chromatography followed by size exclusion chromatography. A proper folding of chimeras can be probed using conformational-specific antibodies that can target γ and δ variable domains. Chimeras can be used in antibody dependent cell mediated cytotoxicity (ADCC) and complement dependent cytotoxicity (CDC) assays to determine functional efficacy. After performing in vitro assays, functional efficacy of TCR-antibody chimeras can be tested in vitro and/or in vivo.

In a further aspect, the disclosure also relates to a conjugate comprising the δT-cell receptor chain or a part thereof as defined above which is linked to an agent. The invention also relates to a γT-cell receptor chain or a part thereof as defined above which is linked to an agent. The type of agent used depends from the type of applications envisaged. Such conjugates may be linked to substrates (e.g. chemicals, nanoparticles) and may be used e.g. to deliver chemotherapy to a target of interest. In addition, in diagnostics expression of defined ligands may be tested by taking advantage of the soluble TCRs linked to fluorochromes which are then used as staining tool or for the biochemical isolation of the ligand. In an aspect, the agent is selected from the group consisting of a diagnostic agent, a therapeutic agent, an anti-cancer agent, a chemical, a nanoparticle, a chemotherapeutic agent a fluorescent protein or an enzyme whose catalytic activity could be detected. In some aspects the conjugate may be used as a theragnostic agent.

In one aspect, the fluorescent protein can be selected from the group consisting of: green fluorescent protein (GFP), yellow fluorescent protein (YFP), red fluorescent protein (RFP), Blue fluorescent protein (BFP, Heim R., et al. (1994), Proc. Natl. Acad. Sci., 20; 91 (26): 12501-12504, and Heim R., et al (1996) Curr. Biol., 1; 6(2):178-182), a cyan fluorescent variant known as CFP (Heim R., et al. (1996) supra; Tsien R., et al, (1998) Annu. Rev. Biochem., 67: 509-544); a yellow fluorescent variant known as YFP (Ormo M., et al. (1996), Science, 6; 273(5280): 1392-1395; Wachter R. M., et al. (1998), Structure. 1998 Oct. 15; 6(10):1267-77. doi: 10.1016/s0969-2126(98)00127-0. PMID: 9782051); a violet-excitable green fluorescent variant known as Sapphire (Tsien 1998; Zapata-Hommer et al. (2003), BMC Biotechnol. 2003 May 22; 3:5. doi: 10.1186/1472-6750-3-5. Epub 2003 May 22. PMID: 12769828; PMCID: PMC161811); Td Tomato (Shaner N. C., et al. (2004) Nat Biotechnol. 2004 December; 22(12):1567-72. doi: 10.1038/nbt1037. Epub 2004 Nov. 21. PMID: 15558047); a cyan-excitable green fluorescing variant known as enhanced green fluorescent protein (eGFP) (Yang Te-Tuan, et al. (1996), Nucleic Acids Research, Volume 24, Issue 22, 1 Nov. 1996, Pages 4592-4593). The presence of a fluorescent protein can be assessed by live cell imaging, flow cytometry, and/or fluorescent spectrophotometry. Fluorescent reporters can be detected using various means including but not limited to microscopy, visual observation, flow cytometry, Luminex, and the like. In an aspect, a fluorescent reporter is detected using flow cytometry.

Each δT-cell (or γT-cell) receptor chain and γδTCR or part thereof defined by reference to their amino acid or encoding nucleic acid sequence is expected to be biologically relevant for designing a medicament for preventing, treating, regressing, curing and/or delaying cancer since each of these chains and γδTCR or part thereof exhibits an anti-tumor activity/response against ovarian tumor.

Vectors

In some aspects the current disclosure also encompasses vectors that facilitate transfer of nucleic acids encoding the disclosed receptors between cells, such as, but not limited to, plasmids, transposons, cosmids, chromosomes, artificial chromosomes, viruses, virions, and the like. A vector may also be a chemical vector, such as a lipid complex or naked DNA. In some aspects the vector may be a viral vector. A viral vector may comprise an expression construct as described herein.

A viral vector or a gene therapy vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2:308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein.

A viral vector and/or a gene therapy vector may be an adenoviral vector, an adeno-associated viral vector or a retroviral vector.

A particularly suitable vector includes an Adenoviral and Adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications. (Russell, 2000, J. Gen. Virol. 81: 2573-2604; Goncalves, 2005, Virol J. 2(1):43) as indicated above. AAV vectors are even more preferred since they are known to result in very stable long-term expression of transgene expression (up to 9 years in dog (Niemeyer et al, Blood. 2009 Jan. 22; 113(4):797-806) and ˜2 years in human (Nathwani et al, N Engl J Med. 2011 Dec. 22; 365(25):2357-65, Simonelli et al, Mol Ther. 2010 March; 18(3):643-50. Epub 2009 Dec. 1)). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34.

Another suitable vector includes a retroviral vector. A preferred retroviral vector for application in the present invention is a lentiviral based viral vector. Lentiviral vectors have the ability to infect and to stably integrate into the genome of dividing and non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

In one aspect, the vector is a lentiviral vector, such as that having the sequence of SEQ ID. NO. 49. In another aspect, a single bicistronic viral vector is used. By way of non-limiting example, a single bicistronic lentiviral vector with a 2A self-cleaving peptide sequence, as set forth in SEQ ID NO: 50, is used as in the experimental section (Xu Y., et al (2019), Cancer Immunology, Immunotherapy, 68: 1979-1993 and Pincha M., et al, (2011), Gene Therapy, 18: 750-764).

TABLE F
Viral Vector Sequences

| SEQ | | | |
|---|---|---|---|
| ID. | | | |
| NOS | Name | Sequence | Type |
| 49 | Lentiviral | aatgtagtcttatgcaatactcttg | NA |
| | vector | tagtcttgcaacatggtaacgatga | |
| | (pLenti | gttagcaacatgccttacaaggaga | |
| | 6.3) | gaaaaagcaccgtgcatgccgattg | |
| | | gtggaagtaaggtggtacgatcgtg | |
| | | ccttattaggaaggcaacagacggg | |
| | | tctgacatggattggacgaaccact | |
| | | gaattgccgcattgcagagatattg | |
| | | tatttaagtgcctagctcgatacat | |
| | | aaacgggtctctctggttagaccag | |
| | | atctgagcctgggagctctctggct | |
| | | aactagggaacccactgcttaagcc | |
| | | tcaataaagcttgccttgagtgctt | |
| | | caagtagtgtgtgcccgtctgttgt | |
| | | gtgactctggtaactagagatccct | |
| | | cagacccttttagtcagtgtggaaa | |
| | | atctctagcagtggcgcccgaacag | |
| | | ggacttgaaagcgaaagggaaacca | |
| | | gaggagctctctcgacgcaggactc | |
| | | ggcttgctgaagcgcgcacggcaag | |
| | | aggcgaggggcggcgactggtgagt | |
| | | acgccaaaaattttgactagcggag | |
| | | gctagaaggagagagatgggtgcga | |
| | | gagcgtcagtattaagcgggggaga | |
| | | attagatcgcgatgggaaaaaattc | |
| | | ggttaaggccagggggaaagaaaaa | |
| | | atataaattaaaacatatagtatgg | |
| | | gcaagcagggagctagaacgattcg | |
| | | cagttaatcctggcctgttagaaac | |
| | | atcagaaggctgtagacaaatactg | |
| | | ggacagctacaaccatcccttcaga | |
| | | caggatcagaagaacttagatcatt | |
| | | atataatacagtagcaaccctctat | |
| | | tgtgtgcatcaaaggatagagataa | |
| | | aagacaccaaggaagctttagacaa | |
| | | gatagaggaagagcaaaacaaaagt | |
| | | aagaccaccgcacagcaagcggccg | |
| | | ctgatcttcagacctggaggaggag | |
| | | atatgagggacaattggagaagtga | |
| | | attatataaatataaagtagtaaaa | |
| | | attgaaccattaggagtagcaccca | |
| | | ccaaggcaaagagaagagtggtgca | |
| | | gagagaaaaaagagcagtgggaata | |
| | | ggagctttgttccttgggttcttgg | |
| | | gagcagcaggaagcactatgggcgc | |
| | | agcgtcaatgacgctgacggtacag | |
| | | gccagacaattattgtctggtatag | |
| | | tgcagcagcagaacaatttgctgag | |
| | | ggctattgaggcgcaacagcatctg | |
| | | ttgcaactcacagtctggggcatca | |
| | | agcagctccaggcaagaatcctggc | |
| | | tgtggaaagatacctaaaggatcaa | |
| | | cagctcctggggatttggggttgct | |
| | | ctggaaaactcatttgcaccactgc | |
| | | tgtgccttggaatgctagttggagt | |
| | | aataaatctctggaacagatttgga | |
| | | atcacacgacctggatggagtggga | |
| | | cagagaaattaacaattacacaagc | |
| | | ttaatacactccttaattgaagaat | |
| | | cgcaaaaccagcaagaaaagaatga | |
| | | acaagaattattggaattagataaa | |
| | | tgggcaagtttgtggaattggttta | |
| | | acataacaaattggctgtggtatat | |
| | | aaaattattcataatgatagtagga | |
| | | ggcttggtaggtttaagaatagttt | |
| | | ttgctgtactttctatagtgaatag | |
| | | agttaggcagggatattcaccatta | |
| | | tcgtttcagacccacctcccaaccc | |
| | | cgaggggacccgacaggcccgaagg | |
| | | aatagaagaagaaggtggagagaga | |
| | | gacagagacagatccattcgattag | |
| | | tgaacggatctcgacggtatcggtt | |
| | | aacttttaaaagaaaaggggggatt | |
| | | ggggggtacagtgcaggggaaagaa | |
| | | tagtagacataatagcaacagacat | |
| | | acaaactaaagaattacaaaaacaa | |
| | | attacaaaaattcaaaattttatcg | |
| | | atgcatgctgaaagaccccacctgt | |
| | | aggtttggcaagctagcttaagtaa | |
| | | cgccattttgcaaggcatggaaaat | |
| | | acataactgagaatagagaagttca | |
| | | gatcaaggttaggaacagagagaca | |
| | | gcagaatatgggccaaacaggatat | |
| | | ctgtggtaagcagttcctgccccgg | |
| | | ctcagggccaagaacagatggtccc | |
| | | cagatgcggtcccgccctcagcagt | |
| | | ttctagagaaccatcagatgtttcc | |
| | | agggtgccccaaggacctgaaaatg | |
| | | accctgtgccttatttgaactaacc | |
| | | aatcagttcgcttctcgcttctgtt | |
| | | cgcgcgtttctgctccccgagctca | |
| | | ataaaagagcccacaacccctcact | |
| | | agcggccctaatacgactcactata | |
| | | gcccggctcgaggctaggcgcgaat | |
| | | tcgccacccggatccacgcgtaccg | |
| | | gttagtaatgatcgacaatcaacct | |
| | | ctggattacaaaatttgtgaaagat | |
| | | tgactggtattcttaactatgttgc | |
| | | tccttttacgctatgtggatacgct | |
| | | gctttaatgcctttgtatcatgcta | |
| | | ttgcttcccgtatggctttcatttt | |
| | | ctcctccttgtataaatcctggttg | |
| | | ctgtctctttatgaggagttgtggc | |
| | | ccgttgtcaggcaacgtggcgtggt | |
| | | gtgcactgtgtttgctgacgcaacc | |
| | | cccactggttggggcattgccacca | |
| | | cctgtcagctcctttccgggacttt | |
| | | cgctttccccctccctattgccacg | |
| | | gcggaactcatcgccgcctgccttg | |
| | | cccgctgctggacaggggctcggct | |
| | | gttgggcactgacaattccgtggtg | |
| | | ttgtcggggaagctgacgtcctttc | |
| | | catggctgctcgcctgtgttgccac | |
| | | ctggattctgcgcgggacgtccttc | |
| | | tgctacgtcccttcggccctcaatc | |
| | | cagcggaccttccttcccgcggcct | |
| | | gctgccggctctgcggcctcttccg | |
| | | cgtcttcgccttcgccctcagacga | |
| | | gtcggatctccctttgggccgcctc | |
| | | cccgcctggcgatggtacctttaag | |
| | | accaatgacttacaaggcagctgta | |
| | | gatcttagccactttttaaaagaaa | |
| | | aggggggactggaagggctaattca | |
| | | ctcccaacgaagacaagatctgctt | |
| | | tttgcttgtactgggtctctctggt | |
| | | tagaccagatctgagcctgggagct | |
| | | ctctggctaactagggaacccactg | |
| | | cttaagcctcaataaagcttgcctt | |
| | | gagtgcttcaagtagtgtgtgcccg | |
| | | tctgttgtgtgactctggtaactag | |
| | | agatccctcagacccttttagtcag | |
| | | tgtggaaaatctctagcagtagtag | |
| | | ttcatgtcatcttattattcagtat | |
| | | ttataacttgcaaagaaatgaatat | |
| | | cagagagtgagaggaacttgtttat | |
| | | tgcagcttataatggttacaaataa | |
| | | agcaatagcatcacaaatttcacaa | |
| | | ataaagcatttttttcactgcattc | |
| | | tagttgtggtttgtccaaactcatc | |
| | | aatgtatcttatcatgtctggctct | |
| | | agctatcccgcccctaactccgccc | |
| | | atcccgcccctaactccgcccagtt | |
| | | ccgcccattctccgccccatggctg | |
| | | actaattttttttatttatgcagag | |
| | | gccgaggccgcctcggcctctgagc | |
| | | tattccagaagtagtgaggaggctt | |
| | | ttttggaggcctagggacgtaccca | |
| | | attcgccctatagtgagtcgtatta | |
| | | cgcgcgctcactggccgtcgtttta | |
| | | caacgtcgtgactgggaaaaccctg | |
| | | gcgttacccaacttaatcgccttgc | |
| | | agcacatccccctttcgccagctgg | |
| | | cgtaatagcgaagaggcccgcaccg | |
| | | atcgcccttcccaacagttgcgcag | |
| | | cctgaatggcgaatgggacgcgccc | |
| | | tgtagcggcgcattaagcgcggcgg | |
| | | gtgtggtggttacgcgcagcgtgac | |
| | | cgctacacttgccagcgccctagcg | |
| | | cccgctcctttcgctttcttccctt | |
| | | cctttctcgccacgttcgccggctt | |
| | | tccccgtcaagctctaaatcggggg | |
| | | ctccctttagggttccgatttagtg | |
| | | ctttacggcacctcgaccccaaaaa | |
| | | acttgattagggtgatggttcacgt | |
| | | agtgggccatcgccctgatagacgg | |
| | | tttttcgccctttgacgttggagtc | |
| | | cacgttctttaatagtggactcttg | |
| | | ttccaaactggaacaacactcaacc | |
| | | ctatctcggtctattcttttgattt | |
| | | ataagggattttgccgatttcggcc | |
| | | tattggttaaaaaatgagctgattt | |
| | | aacaaaaatttaacgcgaattttaa | |
| | | caaaatattaacgcttacaatttag | |
| | | gtggcacttttcggggaaatgtgcg | |
| | | cggaacccctatttgtttatttttc | |
| | | taaatacattcaaatatgtatccgc | |
| | | tcatgagacaataaccctgataaat | |
| | | gcttcaataatattgaaaaaggaag | |
| | | agtatgagtattcaacatttccgtg | |
| | | tcgcccttattcccttttttgcggc | |
| | | attttgccttcctgtttttgctcac | |
| | | ccagaaacgctggtgaaagtaaaag | |
| | | atgctgaagatcagttgggtgcacg | |
| | | agtgggttacatcgaactggatctc | |
| | | aacagcggtaagatccttgagagtt | |
| | | ttcgccccgaagaacgttttccaat | |
| | | gatgagcacttttaaagttctgcta | |
| | | tgtggcgcggtattatcccgtattg | |
| | | acgccgggcaagagcaactcggtcg | |
| | | ccgcatacactattctcagaatgac | |
| | | ttggttgagtactcaccagtcacag | |
| | | aaaagcatcttacggatggcatgac | |
| | | agtaagagaattatgcagtgctgcc | |
| | | ataaccatgagtgataacactgcgg | |
| | | ccaacttacttctgacaacgatcgg | |
| | | aggaccgaaggagctaaccgctttt | |
| | | ttgcacaacatgggggatcatgtaa | |
| | | ctcgccttgatcgttgggaaccgga | |
| | | gctgaatgaagccataccaaacgac | |
| | | gagcgtgacaccacgatgcctgtag | |
| | | caatggcaacaacgttgcgcaaact | |
| | | attaactggcgaactacttactcta | |
| | | gcttcccggcaacaattaatagact | |
| | | ggatggaggcggataaagttgcagg | |
| | | accacttctgcgctcggcccttccg | |
| | | gctggctggtttattgctgataaat | |
| | | ctggagccggtgagcgtgggtctcg | |
| | | cggtatcattgcagcactggggcca | |
| | | gatggtaagccctcccgtatcgtag | |
| | | ttatctacacgacggggagtcaggc | |
| | | aactatggatgaacgaaatagacag | |
| | | atcgctgagataggtgcctcactga | |
| | | ttaagcattggtaactgtcagacca | |
| | | agtttactcatatatactttagatt | |
| | | gatttaaaacttcatttttaattta | |
| | | aaaggatctaggtgaagatcctttt | |
| | | tgataatctcatgaccaaaatccct | |
| | | taacgtgagttttcgttccactgag | |
| | | cgtcagaccccgtagaaaagatcaa | |
| | | aggatcttcttgagatccttttttt | |
| | | ctgcgcgtaatctgctgcttgcaaa | |
| | | caaaaaaaccaccgctaccagcggt | |
| | | ggtttgtttgccggatcaagagcta | |
| | | ccaactctttttccgaaggtaactg | |
| | | gcttcagcagagcgcagataccaaa | |
| | | tactgttcttctagtgtagccgtag | |
| | | ttaggccaccacttcaagaactctg | |
| | | tagcaccgcctacatacctcgctct | |
| | | gctaatcctgttaccagtggctgct | |
| | | gccagtggcgataagtcgtgtctta | |
| | | ccgggttggactcaagacgatagtt | |
| | | accggataaggcgcagcggtcgggc | |
| | | tgaacggggggttcgtgcacacagc | |
| | | ccagcttggagcgaacgacctacac | |
| | | cgaactgagatacctacagcgtgag | |
| | | ctatgagaaagcgccacgcttcccg | |
| | | aagggagaaaggcggacaggtatcc | |
| | | ggtaagcggcagggtcggaacagga | |
| | | gagcgcacgagggagcttccagggg | |
| | | gaaacgcctggtatctttatagtcc | |
| | | tgtcgggtttcgccacctctgactt | |
| | | gagcgtcgatttttgtgatgctcgt | |
| | | caggggggcggagcctatggaaaaa | |
| | | cgccagcaacgcggcctttttacgg | |
| | | ttcctggccttttgctggccttttg | |
| | | ctcacatgttctttcctgcgttatc | |
| | | ccctgattctgtggataaccgtatt | |
| | | accgcctttgagtgagctgataccg | |
| | | ctcgccgcagccgaacgaccgagcg | |
| | | cagcgagtcagtgagcgaggaagcg | |
| | | gaagagcgcccaatacgcaaaccgc | |
| | | ctctccccgcgcgttggccgattca | |
| | | ttaatgcagctggcacgacaggttt | |
| | | cccgactggaaagcgggcagtgagc | |
| | | gcaacgcaattaatgtgagttagct | |
| | | cactcattaggcaccccaggcttta | |
| | | cactttatgcttccggctcgtatgt | |
| | | tgtgtggaattgtgagcggataaca | |
| | | atttcacacaggaaacagctatgac | |
| | | catgattacgccaagcgcgcaatta | |
| | | accctcactaaagggaacaaaagct | |
| | | ggagctgcaagctt | |
| 50 | Bicistronic | atggtgtccctgctgcacgccagca | |
| | Lentiviral | ccctggccgtgctgggcgccctgtg | |
| | vector | cgtgtatggcgccggacacctggaa | |
| | (CL5 | cagccccagatcagcagcaccaaga | |
| | full | ccctgagcaagaccgccaggctgga | |
| | DNA, | atgcgtggtgtccggcatcaccatc | |
| | gamma | agcgccacctccgtgtactggtaca | |
| | 2A | gagagagacccggcgaggtcatcca | |
| | delta) | gttcctggtgtccatcagctacgac | |
| | | ggcaccgtgcggaaagagagcggca | |
| | | tccccagcggcaagttcgaggtgga | |
| | | cagaatccccgagaccagcacctcc | |
| | | accctgaccatccacaacgtggaga | |
| | | agcaggacatcgccacctactactg | |
| | | cgccctgtgggagatccaggaactg | |
| | | ggcaagaaaatcaaggtgttcggcc | |
| | | ctggcaccaagctgatcatcaccga | |
| | | caagcagctggacgccgacgtgagc | |
| | | cccaagcctaccatcttcctgccca | |
| | | gcatcgccgagaccaagctgcagaa | |
| | | ggccggcacctacctgtgcctgctg | |
| | | gaaaagttcttccccgacgtgatca | |
| | | agatccactgggaggaaaagaagag | |
| | | caacaccatcctgggcagccaggaa | |
| | | ggcaataccatgaaaaccaacgaca | |
| | | cctacatgaagttcagctggctgac | |
| | | cgtgcccgagaagagcctggacaaa | |
| | | gagcacagatgcatcgtccggcacg | |
| | | agaacaacaagaacggcgtggacca | |
| | | ggaaatcatcttcccccccatcaag | |
| | | accgatgtgatcacaatggacccca | |
| | | aggacaactgcagcaaggacgccaa | |
| | | cgataccctgctgctgcagctgacc | |
| | | aacaccagcgcctactacatgtatc | |
| | | tcctgctgctgctgaagagcgtggt | |
| | | gtacttcgccatcatcacctgctgt | |
| | | ctgctgcggcggaccgccttctgct | |
| | | gcaacggcgagaagagcggcagcgg | |
| | | cgaaggccgcggcagcctgctgacc | |
| | | tgcggcgatgtggaagaaaaccctg | |
| | | gcccgatggagcggatcagcagcct | |
| | | gatccacctgagcctgttctgggcc | |
| | | ggagtgatgagcgccatcgagctgg | |
| | | tgcccgagcaccagaccgtgcccgt | |
| | | gagcatcggcgtgcccgccaccctg | |
| | | cggtgcagcatgaagggcgaggcca | |
| | | tcggcaactactacatcaactggta | |
| | | cagaaagacccagggcaacaccatg | |
| | | accttcatctaccgggagaaggaca | |
| | | tctacggccctggcttcaaggacaa | |
| | | cttccagggcgacatcgacatcgcc | |
| | | aagaacctggccgtgctgaagatcc | |
| | | tggcccccagcgagagggacgaggg | |
| | | cagctactactgcgcctgcgacgcc | |
| | | ctgaagagaaccgacaccgacaagc | |
| | | tgatcttcggcaagggcacccgggt | |
| | | gaccgtggagcccagaagccagccc | |
| | | cacaccaagcccagcgtgttcgtga | |
| | | tgaagaacggcaccaacgtggcctg | |
| | | cctggtgaaagagttctaccccaag | |
| | | gacatccggatcaacctggtgtcca | |
| | | gcaagaagatcaccgagttcgaccc | |
| | | cgccatcgtgatcagccccagcggc | |
| | | aagtacaacgccgtgaagctgggca | |
| | | agtacgaggacagcaacagcgtgac | |
| | | ctgcagcgtgcagcacgacaacaag | |
| | | accgtgcacagcaccgacttcgagg | |
| | | tgaaaaccgactccaccgaccacgt | |
| | | gaagcccaaagagaccgagaacacc | |
| | | aagcagcccagcaagagctgccaca | |
| | | agcccaaggccatcgtgcacaccga | |
| | | gaaggtgaacatgatgagcctgacc | |
| | | gtgctgggcctgcggatgctgttcg | |
| | | ccaagacagtggccgtgaacttcct | |
| | | gctgaccgccaagctgttcttcctg | |
| | | tga | |

Other suitable viral and/or gene therapy vectors include a herpes virus vector, a polyoma virus vector or a vaccinia virus vector.

A viral and/or gene therapy vector comprises a nucleotide encoding a δTCR (or γTCR) chain, or a γδTCR whereby each of said nucleotide sequence is operably linked to the appropriate regulatory sequences. Such regulatory sequence will at least comprise a promoter sequence. Suitable promoters for expression of such a nucleotide sequence from gene therapy vectors include e.g. cytomegalovirus (CMV) intermediate early promoter, viral long terminal repeat promoters (LTRs), such as those from murine moloney leukaemia virus (MMVLV) rous sarcoma virus, or HTLV-1, the simian virus 40 (SV 40) early promoter, the MSCV promoter and the herpes simplex virus thymidine kinase promoter. Exemplary promoter sequences are provided in Table E. Transposon or other non-viral delivery systems may also be used in this context. All systems can be used in vitro or in vivo.

In an embodiment, the polynucleotide further comprises at least one nucleic acid encoding at least one cis-acting regulatory element which facilitates co-expression of the two subunits of the γδT-cell receptor. A cis-acting regulatory element may be selected from a 2A self-cleaving peptide, a 2A-like cis-acting hydrolase element and an IRES sequence (e.g., Picornavirus IRES, Apthovirus IRES, Hepatitis A IRES, Pestivirus IRES, Hepesvirus TRES, and combinations thereof). A 2A self-cleaving peptide may be selected from a T2A, a P2A, an E2A, and an F2A peptide.

In an embodiment, the polynucleotide is multicistronic. The polynucleotide may be, for example, bicistronic.

A viral and/or gene therapy vector may optionally comprise a further nucleotide sequence coding for a further polypeptide. A further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Green (supra).

Immune Cells and Methods of Obtaining Immune Cells

In a further aspect, there is provided a cell comprising the nucleic acid construct or the vector as earlier described herein. In an aspect, the cell is a mammalian cell, preferably a human cell. In an aspect, the cell is an immune cell such as a T cell, an alpha-beta T cell, a gamma-delta T cell, CD4+ T cell, CD8+ T cell, a T effector cell, a lymphocyte, a B cell, an NK cell, an NKT cell, a myeloid cell, a monocyte, a macrophage, or a neutrophil. In an aspect, the cell is a T cell. In a preferred aspect, the T cell is an αβT cell.

In an aspect, such a T cell comprises a nucleic acid molecule encoding the amino acid sequence as identified earlier herein and/or wherein said T cell expresses the amino acid sequence as identified earlier herein and/or wherein said T cell comprises a nucleic acid molecule as identified earlier herein.

T Cells

T cells, or T lymphocytes, belong to a group of white blood cells named lymphocytes, which play a role in cell-mediated immunity. T cells originate from hematopoietic stem cells in the bone marrow, mature in the thymus (that is where the T is derived from), and gain their full function in peripheral lymphoid tissues. During T cell development, CD4⁻CD8⁻ T cells (negative for both the CD4 and CD8 co-receptor) are committed either to an αβ or γδ fate as a result of an initial βTCR or δTCR gene rearrangement. Cells that undergo early β chain rearrangement express a pre-TCR structure composed of a complete β-chain and a pre-TCRα-chain on the cell surface. Such cells switch to a CD4⁺CD8⁺ state, rearrange the TCRα-chain locus, and express a mature αβTCR on the surface. CD4⁻CD8⁻ T cells that successfully complete the γ gene rearrangement before the β-gene rearrangement express a functional γδTCR and remain CD4⁻CD8⁻. (Claudio Tripodo et al. Gamma delta T cell lymphomas Nature Reviews Clinical Oncology 6, 707-717, December 2009)). The T-cell receptor associates with the CD3 protein complex. Mature T cells, i.e. expressing a αβTCR or a γδTCR, express the T-cell receptor complex on the cell surface. The γδT-cells, which constitute about 1-5% of the total population of T cells, can be divided in further subpopulations which, in humans, is based on TCRδ-chain expression. Within the extracellular domain of a T-cell receptor three complementarity determining regions (CDR1, CDR2, CDR3) are located. These regions are in general the most variable domains and contribute significantly to the diversity among TCRs. CDR regions are composed during the development of a T cell where so-called Variable-(V), Diverse-(D), and Joining-(J)-gene segments are randomly combined to generate diverse TCRs. Of the three CDR regions CDR3, for both αβT cells and γδT cells, is the most variable one, and is therefore the key player in antigen/ligand recognition.

αβT Cells

αβT cells may be defined with respect to function as T lymphocytes that express an αβTCR, which recognize peptides bound to MHC molecules (major histocompatibility complex), which are expressed on the surface of various cells. MHC molecules present peptides derived from the proteins of a cell. When for example a cell is infected with a virus, the MHC will present viral peptides, and the interaction between the αβTCR on the T cell and the MHC-complex on the target cell (i.e. the virus infected cell) activates specific types of T cells which initiate and immune responses to eliminate the infected cell. Hence, αβT cells may be functionally defined as being cells capable of recognizing peptides bound to MHC molecules. αβT cells may be selected from peripheral blood for example via the CD3 antigen as described below and, in the examples, as the large majority of T cells have the αβTCR. αβT cells may also be selected with an antibody specific for the αβTCR, such as described below. From such selected cells, the nucleic acid (or amino acid) sequence corresponding to the αT-cell receptor chain and the βT-cell receptor chain may be determined by sequencing, preferably as carried out in the experimental part. Hence, αβT cells may also be defined as being cells comprising a nucleic acid (or amino acid) sequence corresponding to the αT-cell receptor chain and/or the βT-cell receptor chain. In an aspect, αβT cells expresses an αβTCR.

γδT Cells

γδT cells may be functionally defined in that they are specifically and rapidly activated by e.g., a set of non-peptidic phosphorylated isoprenoid precursors, collectively named phosphoantigens or stress signals medicated by non-classical HLA molecules like CD1 (this is the case for the Vγ9Vδ2 T cell subset). Phosphoantigens are produced by virtually all living cells, though the levels are usually very low in healthy cells, and increased in transformed/malignant cells or cells infected with e.g. Mycobacterium tuberculosis, which deliver a derivate of phosphoantigens. Activation of γδT cells comprises clonal expansion, cytotoxic activity and expression and release of cytokines. γδT cells are also defined by expression of the γδT cell receptor. For example, cells may be selected using an antibody specific for the γδT cell receptor such as described below. From such selected cells, the nucleic acid (or amino acid sequence) sequence corresponding to the γT-cell receptor chain and/or the δT-cell receptor chain may be determined by sequencing, preferably as carried out in the experimental part. Hence, γδT cells may also be defined as being cells naturally comprising a nucleic acid (or amino acid) sequence corresponding to a γT-cell receptor chain and/or a δT-cell receptor chain. In an aspect, γδT cells expresses an γδTCR.

The person skilled in the art is well capable of selecting and/or identifying cell populations characterized by expression of an antigen or receptor on the surface of the cell such as described throughout herein. It is understood that with regard to expression on the surface of cells, such as CD3, CD4, CD8, αβTCR, and γδTCR, this is typically done in a population of cells of which a portion of cells have a much higher level of expression of the antigen when compared to cells having a lower level of expression. Hence, the terms positive or negative are to be understood as being relative, i.e. positive cells have a much higher expression level as compared to cells being negative. Cells being negative in this sense may thus still have an expression level which may be detected.

Expression on the surface of cells may be analyzed using Fluorescence Activated Cell Sorting (FACS), and many specific antibodies are commercially available, e.g. such as for CD3, CD4, CD8, αβTCR, γδTCR, δ1TCR and δ2TCR that are suitable for such FACS analysis, such as described in the examples and as available. As an example, αβT cells can also be defined and selected as being positive for αβTCR in FACS. The same holds for γδT cells. Antibodies suitable for FACS or similar separation techniques (such as e.g. antibodies conjugated to magnetic beads) are widely available. Conditions are selected, such as provided by the antibody manufacturer that allows the selection of negative and/or positive cells.

Examples of antibodies that may be suitable for selection of γδ-T cells, or engineered γδT cells such as available from BD Pharmingen (BD, Franklin Lakes, NJ USA) is Vδ2-FITC (clone B6, #555738), or such as from Thermofisher Scientific (Waltham, MA USA) is Vγ1-PE-Cy7 (clone TS8.2, #25-5679-42), or such as available from Biolegend (San Diego, CA, USA) is αβTCR-BV785 (clone IP26, #306742) or such as available from Beckman Coulter (Brea, CA, USA) is pan-γδTCR-PE (clone IMMU510, #IM1418U), or such as available from Miltenyi Biotec (Bergisch Gladbach, Germany) is CD3-VioGreen (clone REA613, #130-113-142). Similarly, suitable antibodies for αβT cell depletion/selection, such as anti-Biotin αβTCR (clone IP26, Biolegend, San Diego, CA, USA, #306704).

Accordingly, the present disclosure provides T cells. The T cells may be primary cells, for example from a subject, such as described in the examples for a human subject. The T cells may be αβ or γδT cells derived from a human subject. Alternatively, the T-cells may be T cell lines, such as SupT-1 or Jurkat cells or any other widely available cell line. Any cell type, being a primary cell or any cell line will suffice, as long as the cell population, or a substantial part thereof, expresses the T-cell receptor, i.e. such as being positive for the αβTCR or the γδTCR in a FACS sorting or the like as described above, such a cell population may be contemplated. Also, any cell or cell population may be contemplated that, when provided with a γδTCR according to the invention is capable of forming a functional TCR complex and exerting e.g. a functional cytotoxic response and/or cytokine production as later defined herein. The cell that is provided may also be a progenitor cell, preferably a blood progenitor cell such as a thymocyte or a blood stem cell, which after it has been provided with the right stimuli can develop into T cells.

Preferably, T cells provided express or are able to express a γδTCR. T cells may have been transduced to express a γδTCR or already express a γTCR and have been transduced to express a δTCR (or respectively already express a δTCR and have been transduced to express a γTCR), comprising the nucleic acid sequences encoding the sequence as earlier identified herein). All theoretical combinations of a γ- with a δ-chain of the TCR provided herein are encompassed.

In order to validate the biological relevance of the γT-cell and/or δT-cell receptor chain and/or the γδTCR, the anti-tumor activity/response of a T cell expressing a defined nucleic acid molecule encoding an amino acid sequence as defined herein is determined. The T cell may already express a δT-cell (or γT-cell) receptor chain identified herein. It is clear that the biological relevance of a δT-cell (or a γT-cell respectively) receptor chain may only be assessed when a T cell is transduced with (or expresses) a δT-cell (or a γT-cell respectively) receptor chain. In an aspect, an anti-tumor activity/response of such sequence is assessed in a T cell that does not endogenously express a gamma or delta chain of the TCR on their cell surface. Such a cell may be an αβT cell or a NK cell.

The nucleic acid sequences encoding the δT-cell receptor, for example the δ2-cell receptor chain, may be introduced into T cells to provide an engineered T cell as explained in the general part of the description dedicated to the definitions.

Alternatively or in combination with, a nucleic acid sequence encoding a γT-, for example γ9T-cell receptor chain, may be introduced into T cells to provide an engineered T cell.

It is clear to a skilled person that the T cells used should also express a γT-cell receptor chain in order to assess the biological relevance of a δT-cell receptor chain. In other words, a γδTCR is preferably expressed in said T cells, the δTCR being the one identified herein.

It is also clear to a skilled person that the T cells used should express a δT-cell receptor chain in order to assess the biological relevance of a γT-cell receptor chain. In other words, a γδTCR is preferably expressed in said T cells, the γTCR being the one identified herein.

In an exemplary aspect, the nucleic acid molecule encoding the δT-cell (or γT-cell) receptor chain or part thereof is provided in an expression vector or in a retroviral or lentiviral vector in a T cell. This has been extensively explained in the general part of the description dedicated to the definitions.

T cells may be expanded before or after the transfer of the nucleic acids encoding the δT- and/or γT-cell receptor chain. Preferably, the expansion is after the transfer such that the amount of nucleic acids that needs to be transferred is as low as possible. This expansion of T cells may be performed by stimulation with anti-CD3/CD28 polymeric nanomatrix beads, in the presence of IL-7 and IL-15. The anti-tumor activity/response of the provided T cell expressing a δT-cell (and/or γT-cell) receptor chain may be assessed using any technique known to the skilled person. A δT-cell receptor chain may be a δ1 or a δ3-T-cell receptor chain. A γT-cell receptor chain may be a γ3, γ4 or a γ9T-cell receptor chain.

As soon as an effect can be seen in the assays described herein, one can conclude that the δT-cell and/or γT-cell receptor chain and/or γδTCR exhibits an anti-tumor response or anti-tumor activity. Therefore, as soon as an effect had been seen/determined/assessed on tumor cell division rate, tumor cell death, tumor cell cytolysis/cytotoxicity, binding to the tumor cell, induction of the production of a cytokine such as IFN-γ, IL-2 or TNFα, the δT-cell and/or γT-cell receptor chain and/or γδTCR would be considered to exhibit an anti-tumor response. In these assays, a negative control may be T cells that are untransduced or that are transduced by an empty viral vector or that are transduced by a control δT-cell and/or γT-cell receptor chain.

In one aspect, determining an anti-tumor response or reactivity or activity comprises contacting the T cells with tumor cells or tumor cell lines. Determining an anti-tumor activity may include any assay in which an anti-tumor effect may be determined, such as having an effect on tumor cell division rate, i.e. the speed with which the tumor cells divide, cell death, cytolysis/cytotoxicity of the tumor cell, binding to the tumor cells, induction of the production of a cytokine such as IFN-γ, IL-2 or TNFα.

Tumor cells may be any kind of tumor cells relevant to gynaecological cancers for example ovarian cancer. For example, primary tumor cells from a patient. The tumor cells may be tumor cells from cell lines, such as the cell lines listed hereafter: Caki-2, HT29, SK-OV-3, 769-P, 786-O, COV504, MDA-MB-231, BLM, Hs895.T, SW480, RKO, IgR39D, HAP-1, OVCAR-3, MZ1851RC, which are well known in the art. In some exemplary aspects, the cell lines are ovarian cancer cell lines including but not limited to OVCAR-3, OV-90, COV504, CAOV-3 and/or SK-OV-3. Tumor cell lines may easily be obtained from the American Type Culture Collection (ATCC, Manassas, Virginia) and the like.

In a preferred aspect, determining the anti-tumor responses includes contacting the T cell expressing a defined nucleic acid molecule encoding an amino acid comprising a δT-cell and/or γT-cell receptor chain and/or γδTCR identified herein and measuring its ability to lyse the tumor cell and/or induce the production of a cytokine such as IFN-γ, IL-2 or TNFα. This contacting step may have a duration from 10 hours to 1, 2, 3, 4, 5 days. The ability to lyse the tumor cells include providing a fixed amount of tumor cells with which T cell expressing a defined nucleic acid molecule encoding an amino acid comprising a δT-cell and/or γT-cell receptor chain and/or γδTCR identified herein is contacted and after an incubation period the number of viable tumor cells is counted.

An anti-tumor response may have been identified or determined when the number of viable tumor cells at the end of the incubation step is less than 90%, less than 80%, less than 70%, less than 60%, less than 50%, less than 40%, less than 30%, less than 20%, less than 10% of the number of initial tumor cells at the onset of the incubation step.

Alternatively, an anti-tumor response may have been identified or determined when the number of viable tumor cells at the end of the incubation step with the T cells is lower than the number of tumor cells at the end of a similar incubation/contacting step with control T cells not comprising sequences of the invention. Lower in this context may mean at least 10% lower, at least 20% lower, at least 30% lower, at least 40% lower, at least 50% lower, at least 60% lower, at least 70% lower, at least 80% lower, at least 90% lower.

In addition, or as alternative to the counting of the number of viable tumor cells at the end of the incubation/contacting step, one may also perform a ⁵¹Chromium-release assay which is known to the skilled person. The amount of ⁵¹Chromium release is a measure of the number of cells that have been lysed.

In an aspect, one may assess the cytotoxicity of the δT-cell and/or γT-cell receptor chain and/or of the γδTCR (or the cytotoxicity or T cells expressing them) by incubating T cells expressing them with tumor cell lines at E:T ratio 1:1. Suitable tumor cell lines have been described earlier herein. The incubation may have a duration of 1, 2, 3, 4 days. In an aspect, the duration is 2 days. Control T cells may also be used. Cytotoxicity may be measured by xCELLigence and plotted as percentage of cytolysis relative to maximum cytolysis induced by treatment of the target cells with the detergent Triton-X-100.

In an aspect, when the percentage of cytolysis of δT-cell and/or γT-cell receptor chain and/or of γδTCR and/or of T cells expressing them assessed at the end of the incubation step is higher (preferably at least 20%, at least 30%, at least 40%, at least 50%, at least 60%, at least 70%, at least 80%, at least 90%, at least 100% or more) than the percentage of cytolysis assessed when the same tumor cells are contacted with control T cells, the δT-cell and/or γT-cell receptor chain and/or γδTCR or T cells expressing them is said to exhibit an anti-tumor response.

In an aspect, when the percentage of cytolysis of the δT-cell and/or γT-cell receptor chain and/or γδTCR and/or of T cells expressing them assessed at the end of the incubation step is at least 20%, at least 30%, at least 40%, at least 50%, at least 60%, at least 70%, at least 80%, at least 90%, at least 100%, the δT-cell (and/or γT-cell) receptor chain and/or γδTCR and/or T cells expressing them is said to exhibit an anti-tumor response.

Similarly, the production of a cytokine such as IFN-γ, IL-2 or TNFα or the secretion or the expression of activation markers may also be determined, e.g. via antibody staining, ELISA and/or quantitative PCR for the expressed mRNA. Assays for determining the production of a cytokine such as IFN-γ, IL-2 or TNFα are commercially widely available. When the production of a cytokine such as IL-2, TNFα or IFN-γ is detected at the end of the contacting step, the T cell expressing a δT-cell and/or γT-cell receptor chain and/or γδTCR identified herein is said to exhibit an anti-tumor response. Alternatively and preferably, when the amount of IFN-γ, IL-2 or TNFα produced at the end of the contacting step with said T cells is higher (preferably at least 10%, at least 20%, at least 30%, at least 40%, at least 50%, at least 60%, at least 70%, at least 80%, at least 90%, at least 100% or more) than the amount of IFN-γ, IL-2 or TNFα produced when tumor cells are contacted with control T cells, the T cells is said to exhibit an anti-tumor response.

An anti-tumor response may also be determined by assessing the binding of the T cells expressing a δT-cell and/or γT-cell receptor chain and/or a γδTCR identified herein to the tumor cell after contacting both cells together. Such a contacting step may have a duration from 10 hours to 1, 2, 3, 4, 5 days. When binding of said T cell to the tumor cell is detected at the end of the contacting step, said T cell is said to exhibit an anti-tumor response. Alternatively, and preferably, when the binding of said T cell to said tumor cell at the end of the contacting step is higher (preferably at least 10%, at least 20%, at least 30%, at least 40%, at least 50%, at least 60%, at least 70%, at least 80%, at least 90%, at least 100% or more) than the binding of control T cells (see earlier definition) to the same tumor cell, the T cells is said to exhibit an anti-tumor response.

Population of Cells

In a further aspect, there is provided a population of cells comprising the cell as defined earlier herein. In an aspect, such a cell comprises a nucleic acid molecule A1, B1 or C1 or expresses a γδTCR A, B or C as earlier defined herein.

In an aspect, it means that at least 10%, at least 20%, at least 30%, at least 40%, at least 50%, at least 60%, at least 70%, at least 80%, at least 90%, at least 95%, or 100% of the cells within said population are cells comprising a nucleic acid molecule A1, B1 or C1 or expressing a γδTCR A, B or C as earlier defined herein.

The person skilled in the art is well capable of selecting and/or identifying cell populations characterized by expression of such δT-cell or γT-cell receptor chain or a part thereof or a γδTCR or a part thereof using FACS as explained earlier herein.

In an aspect, said T cell expresses a γδTCR comprising A or B or C:

### A:

a δT-cell receptor chain or part thereof comprising a CDR3 region, said δT-cell receptor chain or part thereof being represented by an amino acid sequence, said amino acid sequence comprising at least 60% sequence identity or similarity with any one of amino acid sequences SEQ ID NO: 31-33,
and/or
a γT-cell receptor chain or part thereof comprising a CDR3 region, said γT-cell receptor chain or part thereof being represented by an amino acid sequence, said amino acid sequence comprising at least 80% sequence identity or similarity with any one of amino acid sequences SEQ ID NO: 34-36; or

### B:

a δT-cell receptor chain or part thereof comprising a CDR3 region, said δT-cell receptor chain or part thereof being represented by an amino acid sequence, said amino acid sequence comprising at least 60% sequence identity or similarity with amino acid sequences SEQ ID NO: 1-3, 7-12, 25-27,
and/or
a γT-cell receptor chain or part thereof comprising a CDR3 region, said γT-cell receptor chain or part thereof being represented by an amino acid sequence, said amino acid sequence comprising at least 80% sequence identity or similarity with amino acid sequences SEQ ID NO: 13-15, 19-22, 28-30; or

### C:

a δT-cell receptor chain or part thereof comprising a CDR3 region, said δT-cell receptor chain or part thereof being represented by an amino acid sequence, said amino acid sequence comprising at least 60% sequence identity or similarity with amino acid sequences SEQ ID NO: 7-12,
and/or
a γT-cell receptor chain or part thereof comprising a CDR3 region, said γT-cell receptor chain or part thereof being represented by an amino acid sequence, said amino acid sequence comprising at least 80% sequence identity or similarity with amino acid sequences SEQ ID NO: 19-22.

Pharmaceutical Compositions

In therapeutic applications, an effective amount of a γTCR or δTCR chain or parts thereof or a γδTCR or nucleic acid construct or viral vector or cell expressing these molecules as defined herein is administered to a subject. In one aspect the present disclosure encompasses methods of treating ovarian cancer using pharmaceutical compositions comprising one or more of a receptor, nucleic acid(s), vector(s), cell(s) and/or population(s) of cells as provided herein. Pharmaceutical composition means a mixture of substances suitable for administering to an individual that includes a pharmaceutical agent.

Pharmaceutical compositions of the present invention comprise an effective amount of one or more molecules (i.e., a polypeptide comprising a γTCR or δTCR chain or parts thereof or a γδTCR or nucleic acid construct or viral vector or cell expressing these molecules as defined herein), optionally dissolved or dispersed in a pharmaceutically acceptable carrier. The phrases “pharmaceutically acceptable” and “pharmacologically acceptable” are used interchangeably to refer to molecular entities and compositions that do not produce or produce acceptable adverse, allergic or other untoward reaction when administered to an animal, such as, for example, a human, as appropriate. Whether certain adverse effects are acceptable is determined based on the severity of the disease. The preparation of a pharmaceutical composition that contains at least one active ingredient will be known to those of skill in the art in light of the present disclosure, as exemplified by Remington's Pharmaceutical Sciences, 18th ed., Mack Printing Company, 1990 (“Remington's Pharma. Sci. 18^{th} ed. 1990”), which is incorporated herein by reference. Moreover, for animal (e.g., human) administration, it will be understood that preparations should meet sterility, pyrogenicity, general safety and purity standards as required by FDA Office of Biological Standards.

In some aspects the pharmaceutical composition comprises a δT-cell receptor chain or a part thereof, a γT-cell receptor chain or a part thereof, a γδTCR or a part thereof, a conjugate, a nucleic acid molecule, a nucleic acid construct, a vector, a cell, a population of cells all as defined earlier herein, preferably for use as a medicament. A δT-cell receptor chain or part thereof is, in non-limiting example, a δ2T-cell receptor chain or part thereof. A γT-cell receptor chain or part thereof is, in non-limiting example, a γ9T-cell receptor chain or part thereof. The medicament is preferably for the prevention, suppression, or treatment of cancer or an infection. Accordingly, the invention also relates to a composition, preferably a pharmaceutical composition comprising a δ2T-cell receptor chain or a part thereof, a γ9T-cell receptor chain or a part thereof, a γ9δ2TCR or a part thereof, a conjugate, a nucleic acid molecule, a nucleic acid construct, a vector, a cell or a population of cells all as defined earlier herein. In any of these aspects, pharmaceutical compositions may comprise additional one or more pharmaceutically acceptable carriers, excipients, or other pharmaceutical active agents and any combination thereof.

In some cases, populations of engineered T-cells may be formulated for administration to a subject using techniques known to the skilled artisan. Formulations comprising populations of engineered T-cells may include pharmaceutically acceptable excipient(s). A formulation may include one population of engineered T-cells, or more than one, such as two, three, four, five, six or more distinct populations of engineered T-cells.

Pharmaceutically Acceptable Carriers, Excipients and Active Agents

In certain aspects, compositions disclosed herein may further compromise one or more pharmaceutically acceptable diluent(s), excipient(s), and/or carrier(s). Pharmaceutically acceptable diluents, carriers, and excipients can include, but are not limited to, physiological saline, Ringer's solution, phosphate solution or buffer, buffered saline, and other carriers known in the art. Pharmaceutically acceptable carriers include any and all solvents, adjuvants, dispersion media, coatings, surfactants, antioxidants, preservatives (e.g., antibacterial agents, antifungal agents), isotonic agents, absorption delaying agents, salts, drugs, drug stabilizers, gels, binders, excipients, disintegration agents, lubricants, sweetening agents, flavoring agents, dyes, colorants, other medicinal or pharmaceutical agents, wetting agents, emulsifying agents, solution promoters, solubilizers, antifoaming agents, and such like materials and any combinations thereof, as would be known to one of ordinary skill in the art. (See, e.g., Remington's Pharma. Sci. 18^{th} ed. 1990). Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may also be found for example in Remington's Pharma. Sci. 18^{th} ed. 1990. Except insofar as any conventional carrier is incompatible with the active ingredient, its use in the therapeutic or pharmaceutical compositions is contemplated. In certain embodiments, a pharmaceutical composition described herein comprising a population of cells described herein, further comprises a suitable amount of an antifungal agent. In some cases, a pharmaceutical composition described herein comprises an antifungal agent in an amount sufficient for the pharmaceutical composition to retain at least 20%, 30%, 40%, 50%, 60%, 70%, 80% or 90% of its desired activity for a period of at least 1 month, 2 months, 3 months, 6 months, 1 year, 1.5 years, 2 years, 2.5 years or 3 years.

In certain aspects, pharmaceutical compositions described herein may be formulated in conventional manner using one or more physiologically acceptable carriers comprising excipients and auxiliaries to facilitate processing of engineered cells or vectors into preparations which can be used pharmaceutically. In some aspects, any of the well-known techniques, carriers, and excipients may be used as suitable and/or as understood in the art.

In certain aspects, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, pharmaceutical compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some aspects, pharmaceutical compositions disclosed herein may comprise about 5% to about 99%, about 10%, about 95%, or about 15% to about 90% total amount of polymers as suspending agent(s) by total weight of the composition.

In certain aspects, pharmaceutical compositions disclosed herein may comprise a viscous formulation. In some aspects, viscosity of composition herein may be increased by the addition of one or more gelling or thickening agents. In some aspects, compositions disclosed herein may comprise one or more gelling or thickening agents in an amount to provide a sufficiently viscous formulation to remain on treated tissue. In some aspects, pharmaceutical compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some aspects, pharmaceutical compositions disclosed herein may comprise about 5% to about 99%, about 10%, about 95%, or about 15% to about 90% total amount of gelling or thickening agent(s) by total weight of the composition. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain aspects, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, or at least 50% total amount of one or more agents by total weight of the composition. In some aspects, pharmaceutical compositions disclosed herein may comprise about 5% to about 99%, about 10%, about 95%, or about 15% to about 90% total amount of one or more agents by total weight of the composition. In some aspects, one or more of these agents may be added to improve the performance, efficacy, safety, shelf-life and/or other property of the muscarinic antagonist composition of the present disclosure. In some aspects, additives may be biocompatible, without being harsh, abrasive, and/or allergenic.

In certain aspects, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In some aspects, pharmaceutical compositions disclosed herein may comprise at least 5%, at least 10%, at least 20%, at least 25%, at least 30%, at least 35%, at least 40%, at least 45%, at least 50% total amount of one or more acidifying agents by total weight of the composition. In some aspects, pharmaceutical compositions disclosed herein may comprise about 5% to about 99%, about 10%, about 95%, or about 15% to about 90% total amount of one or more acidifying agents by total weight of the composition.

In certain aspects, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more alkalizing agents by total weight of the composition. In some aspects, pharmaceutical compositions disclosed herein may comprise about 5% to about 99%, about 10%, about 95%, or about 15% to about 90% total amount of one or more alkalizing agents by total weight of the composition.

In certain aspects, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more antioxidants by total weight of the composition. In some aspects, pharmaceutical compositions disclosed herein may comprise about 5% to about 99%, about 10%, about 95%, or about 15% to about 90% total amount of one or more antioxidants by total weight of the composition.

In certain aspects, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more buffering agents by total weight of the composition. In some aspects, pharmaceutical compositions disclosed herein may comprise about 5% to about 99%, about 10%, about 95%, or about 15% to about 90% total amount of one or more buffering agents by total weight of the composition.

In some aspects, the amount of one or more buffering agents may depend on the desired pH level of a composition. In some aspects, pharmaceutical compositions disclosed herein may have a pH of about 6 to about 9. In some aspects, pharmaceutical compositions disclosed herein may have a pH greater than about 8, greater than about 7.5, greater than about 7, greater than about 6.5, or greater than about 6.

In certain aspects, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition. In some aspects, pharmaceutical compositions disclosed herein may comprise about 5% to about 99%, about 10%, about 95%, or about 15% to about 90% total amount of one or more preservatives by total weight of the composition.

In certain aspects, pharmaceutical compositions disclosed herein may comprise one or more surface-acting reagents or detergents. In some aspects, surface-acting reagents or detergents may be synthetic, natural, or semi-synthetic. In some aspects, compositions disclosed herein may comprise anionic detergents, cationic detergents, zwitterionic detergents, ampholytic detergents, amphoteric detergents, nonionic detergents having a steroid skeleton, or a combination thereof. In some aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition. In some aspects, pharmaceutical compositions disclosed herein may comprise about 5% to about 99%, about 10%, about 95%, or about 15% to about 90% total amount of one or more surface-acting reagents or detergents by total weight of the composition.

In certain aspects, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition. In some aspects, pharmaceutical compositions disclosed herein may comprise about 5% to about 99%, about 10%, about 95%, or about 15% to about 90% total amount of one or more stabilizers by total weight of the composition.

In some aspects, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some aspects, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some aspects, pharmaceutical compositions disclosed herein may comprise about 5% to about 99%, about 10%, about 95%, or about 15% to about 90% total amount of one or more tonicity modifiers by total weight of the composition.

In some aspect, the pharmaceutical composition may comprise one or more active agents in addition to the receptor, nucleic acids encoding the receptors, vectors, cells and/or populations of cells provided herein. Non limiting examples of additional active agents include but are not restricted to antibiotics, anti-pyrectics, antimicrobials, antifungals, NSAIDs, chemotherapeutic and anticancer agents.

Chemotherapeutic and anticancer agents commonly used to treat ovarian cancer, and which may be used in combination with any of the compositions disclosed herein include but are not limited to platinum compounds (such as cisplatin or carboplatin), and taxane compounds such as paclitaxel (Taxol®) or docetaxel (Taxotere®). Other chemotherapeutic agents used to treat ovarian cancer and which may be used in combination with any of the compositions disclosed herein, include but are not limited to: Albumin bound paclitaxel (nab-paclitaxel, Abraxane®), Pemetrexed (Alimta®), Irinotecan (CPT-11, Camptosar®), Cyclophosphamide (Cytoxan®), Liposomal doxorubicin (Doxil®), Gemcitabine (Gemzar®), Altretamine (Hexalen®), Ifosfamide (Ifex®), Melphalan (Alkeran), Vinorelbine (Navelbine®), Topotecan (Hycamtin), Etoposide (VP-16), and Capecitabine (Xeloda®).

The actual dosage amount of a composition according to the present disclosure, and the selection of any combination treatment to be administered to an animal or a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### c. Methods

In a further aspect, the present disclosure relates to a method for producing the cells and compositions provided herein and their use in preventing, treating, regressing, curing and/or delaying cancer for example ovarian cancer in a subject wherein a δT-cell receptor chain or a part thereof, a γT-cell receptor chain or a part thereof, a conjugate, a nucleic acid molecule, a nucleic acid construct, a vector, a cell or a population of cells all as defined earlier herein are administered to said subject. A preferred subject is a human being.

In a further aspect, the invention relates to a use of a δ2T-cell receptor chain or a part thereof, a γ9T-cell receptor chain, or a part thereof, a conjugate, a nucleic acid molecule a nucleic acid construct, a vector, a cell or a population of cells all as defined earlier herein for the manufacture of a medicament for preventing, treating, regressing, curing and/or delaying cancer in a subject. A preferred subject is a human being.

Making or Producing Cells

In some aspects the current disclosure encompasses making of pharmaceutical compositions useful in treatment of ovarian cancer. In a particular aspect the current disclosure encompasses production of engineered cells useful in therapy, such cells comprising or capable of expressing a T-cell receptor disclosed herein.

Cells can be obtained from any suitable source for the generation of engineered cells. Cells can be primary cells. Cells can be recombinant cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. ascites, pleural effusion, spleen tissue, and tumors. In certain aspects of the present invention, T-cells can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T-cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In a particular aspect, the engineered cell can be a T-cell. The engineered cell can be an effector (TEFF), effector-memory (TEM), central-memory (TCM), T memory stem (TSCM), naïve (TN), or CD4+ or CD8+ T-cell. The T-cells can also be selected from a bulk population, for example, selecting T-cells from whole blood. The T-cells can also be expanded from a bulk population. The T-cells can also be skewed towards particular populations and phenotypes. The engineered cell can also be expanded ex vivo. The engineered cell can be formulated into a pharmaceutical composition. The engineered cell can be formulated into a pharmaceutical composition and used to treat a subject in need thereof as earlier explained herein. The engineered cell can be autologous to a subject in need thereof. The engineered cell can be allogeneic to a subject in need thereof. The engineered cell can also be a good manufacturing practices (GMP) compatible reagent. The engineered cell can be part of a combination therapy to treat a subject in need thereof. The engineered cell can be a human cell. The subject that is being treated can be a human. Cells can be derived from a healthy donor or from a patient diagnosed with cancer.

Cells can also be obtained from a cell therapy bank. Cells can also be obtained from whole blood, apheresis, or a tumor sample of a subject. A cell can be a tumor infiltrating lymphocytes (TIL). In some cases an apheresis can be a leukapheresis.

A desirable cell population can also be selected prior to modification. A selection can include at least one of: magnetic separation, flow cytometric selection, antibiotic selection. The one or more cells can be any blood cells, such as peripheral blood mononuclear cell (PBMC), lymphocytes, monocytes or macrophages. The one or more cells can be any immune cells such as a lymphocyte, an alpha-beta T-cell, a gamma-delta T-cell, CD4+ T-cell, CD8+ T-cell, a T effector cell, a lymphocyte, a B cell, an NK cell, an NKT-cell, a myeloid cell, a monocyte, a macrophage, or a neutrophil.

In some aspects, the cells can be cultured for extended periods without stimulation or with stimulation. Stimulation may comprise contact with an anti-CD3 antibody or antigen binding fragment thereof immobilized on a surface. Stimulation may comprise contact with a target cell.

For co-stimulation of an accessory molecule on the surface of the T-cells, a ligand that binds the accessory molecule can be used. In some cases a population of T-cells can be CD3-CD28 co-stimulated, for example, contacted with an anti-CD3 antibody and an anti-CD28 antibody, under conditions that can stimulate proliferation of the T-cells.

Conditions appropriate for T-cell culture can include an appropriate media (e.g., Minimal Essential Media or RPMI Media 1640, TexMACS (Miltenyi) or, X-vivo 5, (Lonza)) that may contain factors necessary for proliferation and viability, including serum. In an aspect, cells can be maintained under conditions necessary to support growth; for example, an appropriate temperature (e.g., 37° C.) and atmosphere (e.g., air plus 5% CO2).

In some aspects, the suitable cells can be engineered or modified to express the T-cell receptor disclosed herein and formulated into a pharmaceutical composition. Methods of engineering lymphocytes and immune cells to express a polypeptide of interest are well known in the art. In some aspects the method may comprise genetically modifying the cells to express the receptors provided herein. In some aspects the method may comprise using a viral vector as provided herein. In some aspect the method may comprise using a CRISPR/Cas system as provided herein. More details on obtaining engineered T-cells are provided herein.

A method of attaining suitable cells can comprise sorting cells. In some cases, a cell can comprise a marker that can be selected for the cell. For example, such marker can comprise GFP, a resistance gene, a cell surface marker, an endogenous tag. Cells can be selected using any endogenous marker. Suitable cells can be selected or sorted using any technology. Such technology can comprise flow cytometry and/or magnetic columns. The selected cells can then be infused into a subject. The selected cells can also be expanded to large numbers. The selected cells can be expanded prior to infusion.

Vectors can be delivered to cells ex vivo, such as cells explanted from an individual patient (e.g., lymphocytes, T-cells, bone marrow aspirates, tissue biopsy), followed by re-implantation of the cells into a patient, usually after selection for cells which have incorporated the vector. Prior to or after selection, the cells can be expanded.

Ex vivo cell transfection can also be used for diagnostics, research, or for gene therapy (e.g. via re-infusion of the transfected cells into the host organism). In some cases, cells are isolated from the subject organism, transfected with a nucleic acid (e.g., gene or DNA), and re-infused back into the subject organism (e.g. patient). Further, also in vivo cell transfection can be used for gene therapy, in order to reduced immune reactions of the patient.

In some aspects, populations of engineered T-cells may be formulated for administration to a subject using techniques known to the skilled artisan.

Methods of Treatment

In some aspect the current disclosure is based on the surprising discovery that the compositions provided herein can be used in the treatment of various cancers. In some aspects such cancers are selected from gynecologic cancers (i.e., cancers of the female reproductive system). In some aspects, cancers of the female reproductive system include, but are not limited to, ovarian cancer, cancer of the fallopian tube(s), peritoneal cancer and breast cancer. In some aspects, a gynecologic cancer is associated with homologous recombination repair deficiency/homologous repair deficiency (“HRD”) and/or BRCA1/2 mutation(s). In some aspects, a gynecologic cancer is platinum-sensitive. In some aspects, a gynecologic cancer has responded to a platinum-based therapy. In some aspects, a gynecologic cancer has developed resistance to a platinum-based therapy. In some aspects, a gynecologic cancer has at one time shown a partial or complete response to platinum-based therapy. In some aspects, a gynecologic cancer is now resistant to platinum-based therapy.

In therapeutic applications, an effective amount of a δTCR or a γTCR chain or parts thereof or a γδTCR or a part thereof or nucleic acid molecule or conjugate or nucleic acid construct or viral vector or cell expressing these molecules as defined herein is administered to a subject.

Accordingly, pharmaceutical compositions of the present disclosure comprise an effective amount of one or more molecules (i.e., a δT-cell receptor chain, a γT-cell receptor chain, a γδT-cell receptor, a part thereof, a conjugate, a nucleic acid molecule, a nucleic acid construct, a vector, or a cell such as a T-cell, as described herein), optionally dissolved or dispersed in a pharmaceutically acceptable carrier.

The actual dosage amount of a composition of the present invention administered to an animal or a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Any suitable mode of administration can be used to administer the compositions provided herein in a subject in need thereof. Exemplary modes include, but are not limited to, intravenous injection. Other modes include, without limitation, intratumoural, intradermal, subcutaneous (s.c., s.q., sub-Q, Hypo), intramuscular (i.m.), intraperitoneal (i.p.), intraarterial, intramedullary, intracardiac, intra-articular (joint), intrasynovial (joint fluid area), intracranial, intraspinal, and intrathecal (spinal fluids). Any known device useful for parenteral injection of infusion of the formulations can be used to affect such administration.

For example, the formulations comprising population(s) of engineered T-cells may be administered to a subject using modes and techniques known to the skilled artisan. The formulations comprising population(s) of engineered T-cells that are administered to a subject comprise a number of engineered T-cells that is effective for the treatment and/or prophylaxis of the specific indication or disease. Thus, therapeutically effective populations of engineered T-cells are administered to subjects when the methods of the present invention are practiced. In general, formulations are administered that comprise between about 1×10⁴ and about 1×10¹⁰ engineered T-cells. In most cases, the formulation will comprise between about 1×10⁵ and about 1×10⁹ engineered T-cells, from about 5×10⁵ to about 5×10⁸ engineered T-cells, or from about 1×10⁶ to about 1×10⁷ engineered T-cells. However, the number of engineered T-cells administered to a subject will vary between wide limits, depending upon the location, source, identity, extent and severity of the cancer, the age and condition of the individual to be treated etc. A physician will ultimately determine appropriate dosages to be used.

In some aspects the pharmaceutical compositions and methods provided herein may be administered in conjunction with other anti-cancer therapies for example surgery, chemotherapy, radiation therapy, theragnostic. In some aspects the administration may be done concurrently with other anti-cancer therapies. In some aspects the administration may be done before or after the administration of other anti-cancer therapies.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

It is understood that the examples and embodiments described herein are for illustrative purposes only and that various modifications or changes in light thereof will be suggested to persons skilled in the art and are to be included within the spirit and purview of this application and scope of the appended claims.

Unless specified, reagents employed in the examples are commercially available or can be prepared using commercially available instrumentation, methods, or reagents known in the art. The examples illustrate various aspects of the invention and practice of the methods of the invention. The examples are not intended to provide an exhaustive description of the many different embodiments of the invention. Thus, although the invention has been described in some detail by way of illustration and example for purposes of clarity of understanding, those of ordinary skill in the art will realize readily that many changes and modifications can be made thereto without departing from the spirit or scope of the appended claims.

### Materials & Methods Pertaining to the Examples

γδTCR DNA subcloning and lentivirus preparation: Codon optimized DNA encoding for the full length γ- and δ-chains of four putatively tumor targeting γδTCRs was generated and subcloned by gBlock gene assembly (IDT—Integrated DNA Technologies) into pLenti 6.3 lentiviral bicistronic vector (SEQ ID NO: 50), separated by a T2A self-cleaving peptide. Bicistronic expression of both γ and δTCR chains is driven by a MSCV promoter (SEQ ID NO: 45). This promoter has been disclosed in Jones S., et al (2009) (Joens S., et al (2009), Gene therapy, 20:630-640). Viral genome packaging and transgene expression enhancement are achieved by LTR/Ψ and WPRE regulatory elements, respectively. Lentiviral particles were produced using the LV-Max system from Thermo Fisher Scientific. LV-MAX producer cells (A35827) were transfected with pLenti 6.3 γδTCR transfer construct and packaging mix (pLP1, pLP2, pLP-VSVG). Lentiviral titers were assessed in αβTCR-deficient Jurkat-76 cells by flow cytometry analysis, measuring the percentage of CD3/γδTCR among live cells.

TEG production: TEGs were manufactured starting from αβT-cells enriched by MACS separation from healthy donor leukapheresis material, according to manufacturer instructions. Purified αβT-cells were cultured in TEXMACS medium supplemented with 2.5% human serum (Sanquin), rhIL-7 (20-2000 IU/mL) and rh IL15 (20-200 IU/mL) (both from Miltenyi), and 1% Penicillin/Streptomycin, and activated using TransAct (Miltenyi Biotec) per manufacturer's recommendations. Cells were transduced with γδTCR LV particles (MOI 5) and then expanded for 12 days in TEXMACS medium, 2.5% human serum, rhIL-7 (20-2000 IU/mL) and rh IL15 (20-200 IU/mL), 1% Penicillin/Streptomycin. At the end of the production, transduction efficiency (% γδTCR, >40% in all cases), T-cell purity (>90% in all cases), and relative expression of T-cell markers CD4 and CD8 were measured by flow cytometry. Cells were then cryopreserved in 1 volume of NaCl 0.9%/5% human serum albumin and 1 volume of Cryostor CS10 (Sigma-Aldrich).

Endogenous γ9δ2 T cell production: PBMCs were isolated from a fresh buffy coat by density gradient separation using Ficoll. The γδT cells were directly isolated from PBMCs using anti-TCR γ/δ MicroBead kit (Miltenyi) using the autoMACS (Miltenyi). After isolation (day 0), the γδT cells were activated with CD3/CD28 TransAct (Miltenyi), or with 1 μg/mL coated antibody anti-γδTCR clone B1 (Biolegend) and anti-CD28 (ThermoFisher), and cultured in TexMACS (Miltenyi) medium with Penicillin/Streptomycin (0.5%), human serum (2.4%), IL-2 (20-200 IU/ml), and IL-15 (20-500 IU/ml) (T cell medium). After 5 days of activation, cells were counted and seeded at 0.35-0.4E6 cells per well, activated with the same procedure as day 0, and transduced with the selected lentiviral vectors at a prior set multiplicity of infection (MOI; 10) by diluting the lentivirus in T cell medium. Lentiviral titer was determined as described in lentiviral preparation

Organoid culture: Culture conditions for human ovarian organoids have been described previously (1-4). The basement membrane extract used for expanding colon, breast and models was Matrigel while BME was used for culturing ovarian, organoid models. Minor deviations from literature to the culture media to remove as many undefined components possible. Mainly, in the culture recipe R-Spondin and Noggin conditioned medium were replaced by recombinant (purified) proteins. To activate Wnt signalling, Wnt3A conditioned media was used.

Organoid and TEG co-culture: Organoids were expanded under normal culture conditions. Before the start of the co-culture experiment, organoids were collected and size selected by filtering: 20-100 μm. Tissue culture 96-well plates were used that were precoated with 30 μl/well BME/Matrigel at 37° C. for one hour before plating the T cells. TEGs were added in presence or absence of 10 μM pamidronate (PAM) containing TEG medium followed by the organoids. Each experiment was performed in quadruplet technical replicates per condition.

xCELLigence cytotoxicity assay: TEG anti-tumour activity towards several tumour cell lines was evaluated in vitro by measuring the killing of tumour target cells in a xCELLigence co-culture assay (Agilent, Santa Clara, CA, USA). First, cell lines were harvested, counted and seeded to the appropriate number of cells per well in triplicate in 96 well E-plates, and then placed in the xCELLigence cradles. Target cell adhesion and proliferation was measured for 24 hours. TEG or negative control untransduced αβT-cells were then harvested, counted, resuspended in IMDM medium, 5% human serum, and 1% Penicillin/Streptomycin, and added to the tumour target cells at Effector/Target ratio of 1:1. Loss of target cell adherence, as a readout for cytotoxicity, was measured for 48 hours. Cytotoxicity was calculated as percentage of cytolysis relative to maximum cytolysis induced by treatment of the target cells with the detergent Triton-X-100.

IFN-γ ELISA assay: Cell culture supernatants from xCELLigence cytotoxicity assays were harvested at the end of the 48-72 hour co-culture to measure IFN-γ secretion using a commercial Human IFN-gamma DuoSet ELISA assay (cat nr. DY285B-05, R&D Systems, Minneapolis, MN, US), according to manufacturer's instructions. This is a standard sandwich ELISA using a plate-bound capture antibody and a detection antibody both specific for IFN-γ. The detection antibody is linked to an enzyme which can convert a substrate into an absorbance signal which is measured with a plate reader. The internal standard curve allows absorbance values to be calculated into the IFN-γ concentration (pg/mL) released into the supernatants.

### Example 1

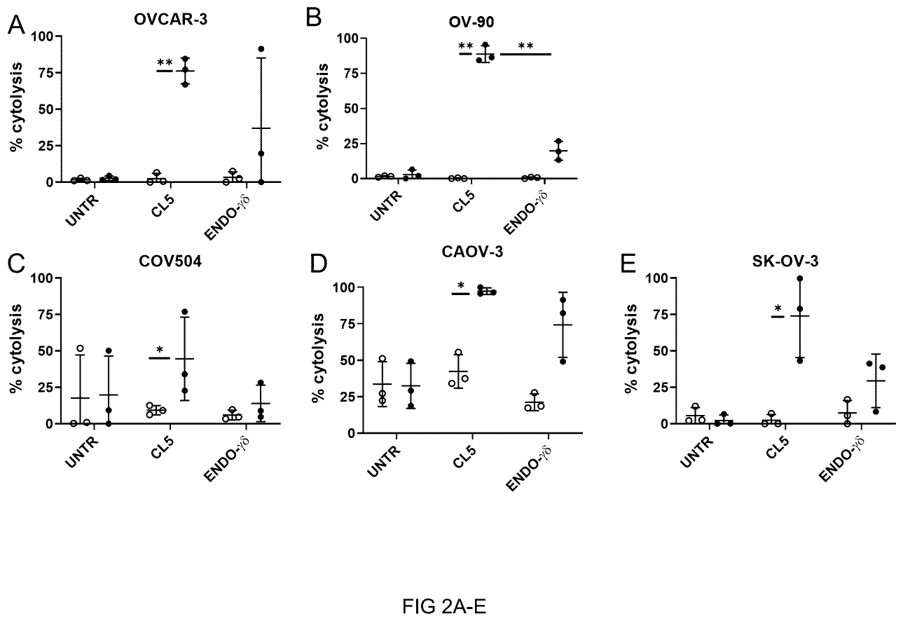

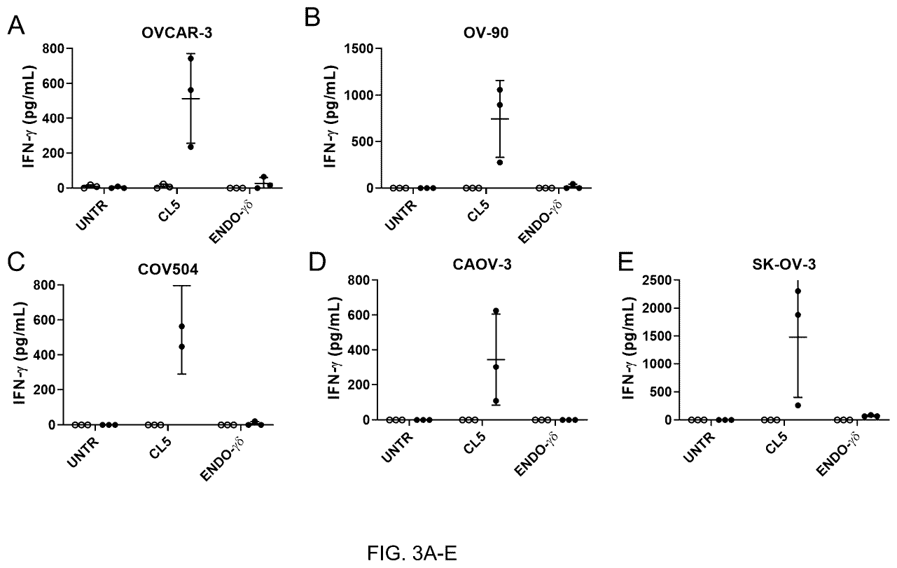

The capacity of αβT-cells engineered to express γ9δ2TCRs (TEGs) to recognize and kill a panel of ovarian tumor cell lines was tested using an impedance-based cytotoxicity assay (FIG. 2A-E). The panel included OVCAR-3, OV-90, COV504, CAOV-3 and SK-OV-3 cell lines. TEG reactivity towards the same tumor cell lines was also assessed by measuring the release of the cytokine IFN-γ into the cell culture supernatants (FIG. 3A-E). TEG expressing CL5 (CDR3 regions represented by SEQ ID NOS: 12, 22) recognized all ovarian tumor cell lines in a PAM dependent manner, in the presence of 10 μM PAM cytolysis was significantly higher compared to absence of PAM.

Reactivity of TEG expressing CL5 against all ovarian tumor cell lines was confirmed by IFN-γ secretion. No IFN-γ secretion was observed in co-cultures with negative control untransduced αβT-cells.

Compared to endogenous γ9δ2 T cells at least similar cytotoxicity and higher IFN-γ secretion was observed for TEG CL5. For OV-90 cell line even significantly improved cytolysis was observed.

In conclusion, TEGs expressing γ9δ2TCRs display a potent and broad anti-tumor reactivity against ovarian tumor cell lines.

### Example 2

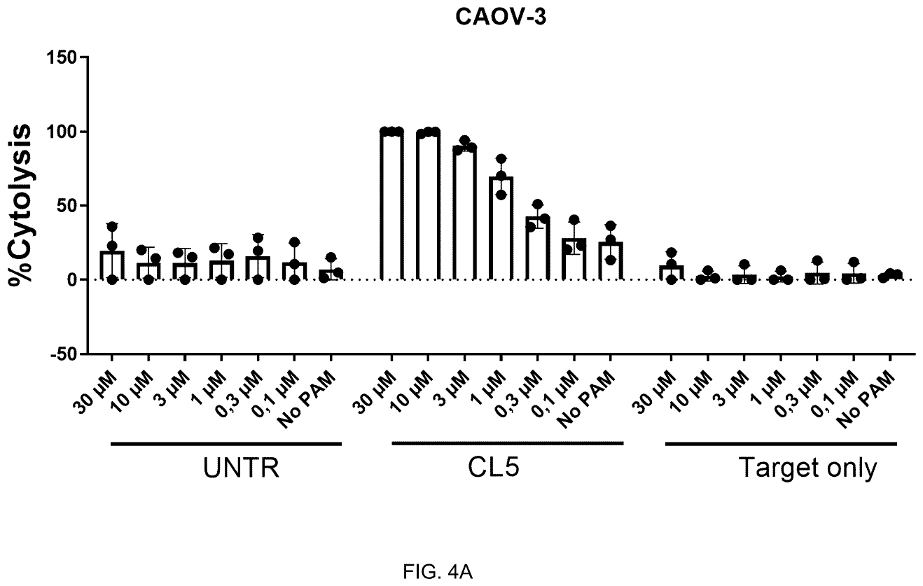

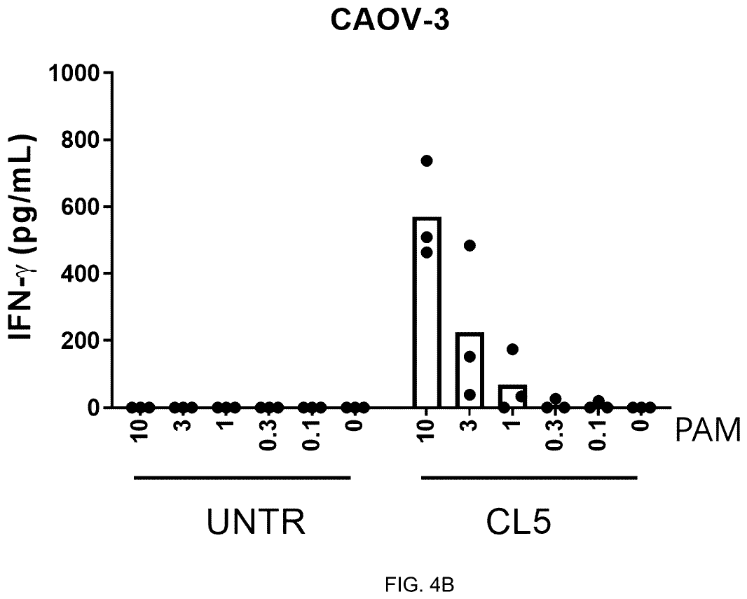

Next, the dose dependency of TEGs expressing γ9δ2TCRs (TEGs) reactivity against ovarian cancer was tested. Cytotoxicity was detected using an impedance-based cytotoxicity assay (FIG. 4A) and release of IFN-γ into the cell culture supernatants (FIG. 4B).

TEGs expressing CL5 (CDR3 regions represented by SEQ ID NOS: 12, 22) were co-cultured with ovarian tumor cell line CAOV-3 with increasing concentration of PAM up to 30 μM. Dose dependent increase in cytotoxicity and IFN-γ release was observed while no effect was detected for the negative control (untransduced αβ T cells, UNTR). Illustrating that TEGs expressing γ9δ2TCRs can have a dose dependent effect when PAM is increased.

### Example 3

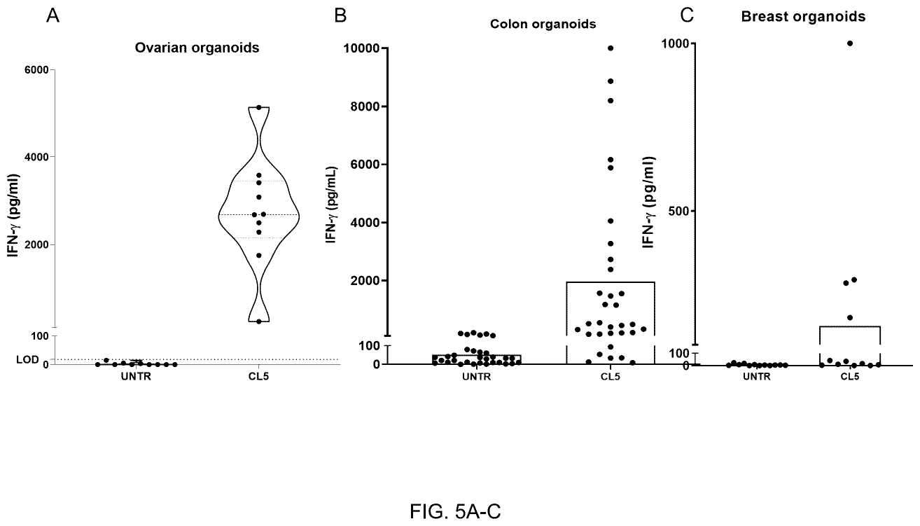

Reactivity of TEGs expressing γ9δ2TCRs against ovarian, colon and breast tumor organoids was tested. TEGs expressing CL5 (CDR3 regions represented by SEQ ID NOS: 12, 22) were co-cultured as described in the method section. After 72 hours release of IFN-γ into the cell culture supernatants was assessed by ELISA. IFN-γ release was detected in all ovarian tumor organoids with 9 out of 10 above 1000 μg/ml (FIG. 5A). None of the co-cultures with the negative control (untransduced αβ T cells, UNTR) showed an IFN-γ release above Limit of Detection (18 pg/ml). Co-cultures of TEGs expressing CL5 with colon (FIG. 5B) and breast (FIG. 5C) tumor organoids showed reactive and non-reactive tumor organoids (signal lower or equal compared to negative control).

This illustrates that ovarian tumor organoids are exceptionally well recognized by TEGs expressing γ9δ2TCRs.

### Example 4

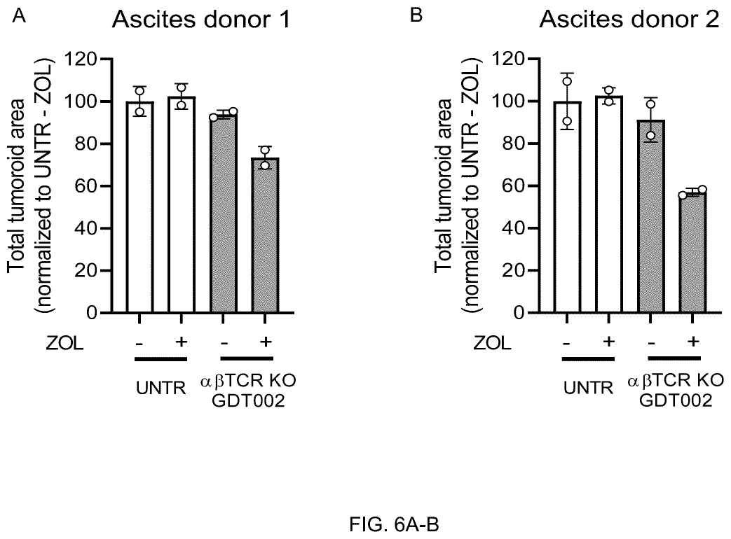

The ability of TRAC (T Cell Receptor Alpha Constant) knockout cells expressing the CL5 (CDR3 regions represented by SEQ ID NOS: 12, 22) to restrict tumor growth was tested in an ascite co-culture experiment. Briefly, a heterogeneous composite of tumor clusters and immune components were isolated from ascites samples from two donors. 2 days before the co-culture, this heterogeneous material was seeded in a hydrogel in a 384 well plate. At the same time, TRAC knock-out αβ T cells expressing or not expressing CL5 (CDR3 regions represented by SEQ ID NOS: 12, 22) were thawed, rested and labeled with cell tracer. At the start of the experiment, the cells expressing CL5 (CDR3 regions represented by SEQ ID NOS: 12, 22) were added to the ascites samples using 40,000 effector cells. The cells were co-cultured for 4 days, in the presents of absence of 5 μM zoledronic acid (ZOL). αβ T cells that were TRAC knock-out and do not express the GDT002 receptor (untransduced αβ T cells, UNTR) were used as a negative control. Total tumoroid volume was used as readout. FIG. 6A-B shows that TRAC knockout cells expressing CL5 (CDR3 regions represented by SEQ ID NOS: 12, 22) were more successful in reducing total tumor area as compared to untransduced cells in two separate ascite donors.

### REFERENCES

1. N. Sachs et al., Long-term expanding human airway organoids for disease modeling. EMBO J. 2019 Feb. 15; 38(4):e100300.
2. E. Driehuis et al., Patient-Derived Head and Neck Cancer Organoids Recapitulate EGFR Expression Levels of Respective Tissues and Are Responsive to EGFR-Targeted Photodynamic Therapy. J Clin. Med. 2019 Nov. 5; 8(11):1880.
3. O. Kopper et al., An organoid platform for ovarian cancer captures intra- and interpatient heterogeneity. Nat Med. 2019 May; 25(5):838-849.
4. T. Sato et al., Long-term expansion of epithelial organoids from human colon, adenoma, adenocarcinoma, and Barrett's epithelium. Gastroenterology 2011 November; 141(5):1762-72.

What is claimed is:

1. A method of treating ovarian cancer in a subject in need thereof, the method comprising: (a) administering to the subject a composition comprising a γ9δ2T-cell receptor or a fragment thereof, a nucleotide sequence encoding it, a vector comprising said nucleotide sequence, or an engineered immune responsive cell that comprises or expresses an exogenous γ9δ2T-cell receptor or a functional fragment thereof and/or (b) contacting an ovarian tumor cell in the subject with the composition.

2. The method of claim 1, wherein the composition is a pharmaceutical composition comprising a pharmaceutically acceptable carrier and/or excipient.

3. The method of claim 1, wherein the engineered immune responsive cell is a αβ T-cell engineered to express said exogenous γ9δ2T-cell receptor or a functional fragment thereof.

4. The method of claim 1, wherein the γ9δ2T-cell receptor or fragment thereof comprises a first polypeptide or the composition comprises a nucleotide sequence encoding the first polypeptide, the first polypeptide having at least 60% identity or similarity with an amino acid sequence selected from SEQ ID NOS: 13-15, 19-22, 24, 28-30, 34-36, and/or the γ9δ2T-cell receptor or fragment thereof comprises a second polypeptide or the composition comprises a nucleotide sequence encoding the second polypeptide, the second polypeptide having at least 60% identity or similarity with an amino acid sequence selected from SEQ ID NOS: 1-3, 7-12, 23, 25-27, 31-33.

5. The method of claim 4, wherein the γ9δ2T-cell receptor comprises a γ9-T-cell receptor chain comprising a γ9-CDR3 region having a sequence selected from any one of SEQ ID NOS: 19-24 and a δ2-CDR3 region having a sequence selected from SEQ ID NOS: 7-12.

6. The method of claim 1, wherein the composition comprises a population of said αβ T-cells engineered to express said γ9δ2T-cell receptor.

7. The method of claim 1, wherein the γ9δ2T-cell receptor comprises a γ9-T-cell receptor chain comprising an amino acid sequence having at least 70% sequence identity or similarity to any one of SEQ ID NOS: 13-15, 19-22, 28-30 or 34-36; and a δ2-T-cell receptor chain comprising an amino acid sequence having at least 70% sequence identity or similarity to any one of SEQ ID NOS: 1-3, 7-12, 25-27 or 31-33.

8. The method of claim 1, wherein the γ9δ2T-cell receptor comprises a γ9-T-cell receptor chain comprising an amino acid sequence having at least 80% sequence identity or similarity to any one of SEQ ID NOS: 13-15, 19-22, 28-30 or 34-36; and a δ2-T-cell receptor chain comprising an amino acid sequence having at least 80% sequence identity or similarity to any one of SEQ ID NOS: 1-3, 7-12, 25-27 or 31-33.

9. The method of claim 1, wherein the γ9δ2T-cell receptor comprises a γ9-T-cell receptor chain comprising an amino acid sequence having at least 90% sequence identity or similarity to any one of SEQ ID NOS: 13-15, 19-22, 28-30 or 34-36; and a δ2-T-cell receptor chain comprising an amino acid sequence having at least 90% sequence identity or similarity to any one of SEQ ID NOS: 1-3, 7-12, 25-27 or 31-33.

10. The method of claim 1, wherein the γ9δ2T-cell receptor comprises a γ9-T-cell receptor chain comprising an amino acid sequence having at least 95% sequence identity or similarity to any one of SEQ ID NOS: 13-15, 19-22, 28-30 or 34-36; and a δ2-T-cell receptor chain comprising an amino acid sequence having at least 95% sequence identity or similarity to any one of SEQ ID NOS: 1-3, 7-12, 25-27 or 31-33.

11. The method of claim 2, wherein the pharmaceutical composition is administered intravenously to the subject.

12. The method of claim 1, wherein administering the composition reduces number of ovarian cancer cells.

13. The method of claim 1, wherein the composition is cytotoxic for ovarian cancer cells.

14. The method of claim 1, wherein administering the composition reduces ovarian cancer growth.

15. The method of claim 1, further comprising administering to the subject an additional anti-cancer therapy.

16. The method of claim 15, wherein the composition is administered to the subject before said administration of the additional anti-cancer therapy.

17. The method of claim 15, wherein the composition is administered to the subject after said administration of the additional anti-cancer therapy.

18. The method of claim 15, wherein the composition is administered to the subject concurrently with administration of additional anti-cancer therapy.

19. The method of claim 15, wherein said additional anti-cancer therapy comprises any one of surgery, radiation, chemotherapy or a combination thereof.

20. The method of claim 1, wherein the subject is a human.
